(19) United States
(12) Patent Application Publication
(10) Pub. No.: US 2024/0290976 A1
(43) Pub. Date: Aug. 29, 2024

(54) CYCLOTETRABENZIL COMPOUNDS FOR ELECTROCHEMICAL LITHIUM-ION STORAGE

(71) Applicant: UNIVERSITY OF HOUSTON SYSTEM, Houston, TX (US)

(72) Inventors: Ognjen Miljanic, Houston, TX (US); Yan Yao, Pearland, TX (US); Said Jalife Jacobo, Houston, TX (US); Judy Wu, Manvel, TX (US); Alexandra Robles, San Francisco, CA (US)

(73) Assignee: UNIVERSITY OF HOUSTON SYSTEM, Houston, TX (US)

(21) Appl. No.: 18/216,410

(22) Filed: Jun. 29, 2023

Related U.S. Application Data
(60) Provisional application No. 63/444,456, filed on Feb. 9, 2023.

(51) Int. Cl. H01M 4/60; C07C 49/792; H01M 10/0525

(52) U.S. Cl. CPC H01M 4/60; C07C 49/792; H01M 10/0525; C07C 2603/92; H01M 2004/028

(57) ABSTRACT

This invention relates to materials for electrodes, to materials for lithium-ion batteries, and to materials for lithium-ion storage.

### CROSS-REFERENCE TO RELATED APPLICATIONS

This application claims the benefit of priority under 35 U.S.C. § 119(e) to U.S. Provisional Patent Application No. 63/444,456 filed Feb. 9, 2023, the contents of which is incorporated herein by reference in its entirety.

### STATEMENT REGARDING FEDERALLY SPONSORED RESEARCH OR DEVELOPMENT

This invention was made with government support under Grant Nos. DMR-1904998 and CHE-1751370 awarded by the National Science Foundation. The government has certain rights in the invention.

### FIELD OF THE INVENTION

In various embodiments, the present invention relates to materials for electrodes, to materials for lithium-ion batteries, and to materials for lithium-ion storage.

### BACKGROUND

Electrochemical energy storage systems such as rechargeable batteries have been regarded as one of the most efficient methods to regulate the output of electricity. Among these systems, state-of-the-art lithium-ion batteries (LIBs) are widely used as power sources for a plethora of mobile electronic devices and electric vehicles. Traditional LIBs based on transition metal oxide cathodes and graphite anodes are approaching their performance limits, and present concerns about the environmental and social impacts of mining materials such as cobalt.

Thus, there is a need in the art for new, environmentally friendly battery materials with tunable properties. The embodiments of the present invention address these and other unfulfilled needs in the art.

### SUMMARY OF THE INVENTION

In various embodiments, the present invention provides an electrode, comprising at least one compound, wherein the at least one compound is selected from the group consisting of a compound of Formula (I), a compound of Formula (III), and a compound of Formula (V), and any combinations thereof, wherein the compound of Formula (I) is:

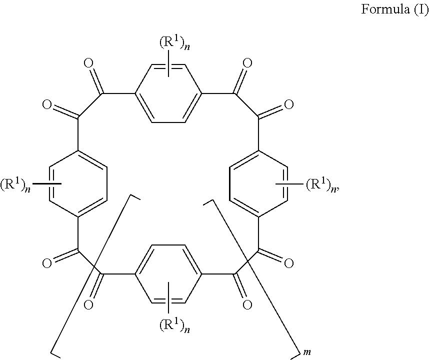

wherein: m is 0, 1, 2, 3, or 4; n is 0, 1, 2, 3 or 4; and R¹ is an electron withdrawing group, or an electron donating group; wherein the compound of Formula (III) is:

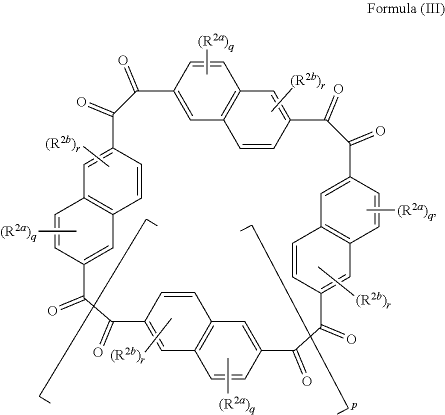

wherein: p is 0, 1, 2, 3, or 4; q is 0, 1, 2, or 3, r is 0, 1, 2, or 3; R^{2a} is an electron withdrawing group, or an electron donating group; and R^{2b} is an electron withdrawing group, or an electron donating group; and wherein the compound of Formula (V) is:

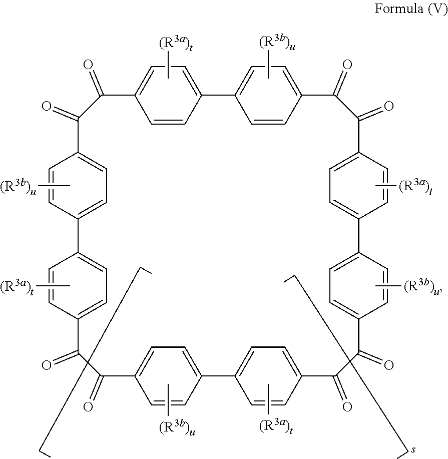

wherein: s is 0, 1, 2, 3, or 4; t is 0, 1, 2, 3 or 4; u is 0, 1, 2, 3, or 4; R^{3a} is an electron withdrawing group, or an electron donating group, and R^{3b} is an electron withdrawing group, or an electron donating group.

In some embodiments, the compound of Formula (I) is a compound of Formula (I-a):

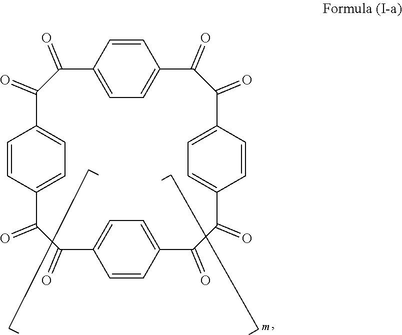

wherein: m is 0, 1, 2, 3, or 4; wherein the compound of Formula (III) is a compound of Formula (III-a):

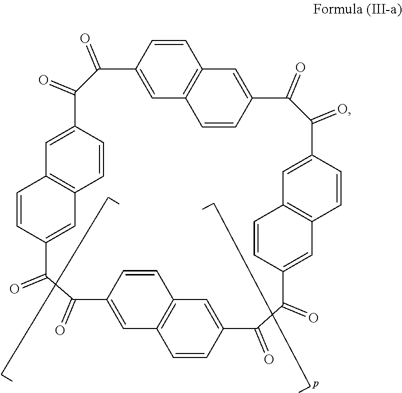

wherein: p is 0, 1, 2, 3, or 4; and wherein the compound of Formula (V) is a compound of Formula (V-a):

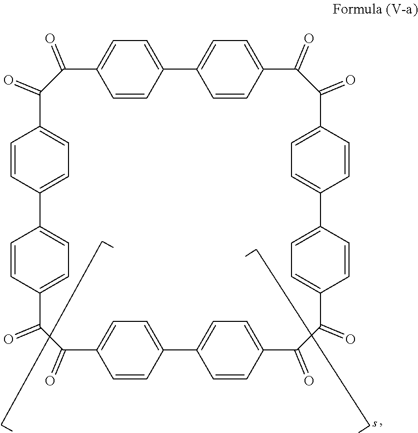

wherein: s is 0, 1, 2, 3, or 4.

In some embodiments, the compound of Formula (I) is.

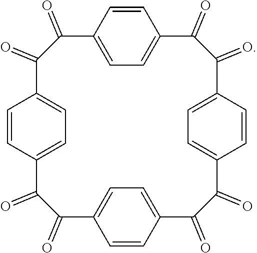 [SMILES: O=C1C(=O)c2ccc(cc2)C(=O)C(=O)c2ccc(cc2)C(=O)C(=O)c2ccc(cc2)C(=O)C(=O)c2ccc1cc2]

In some embodiments, the compound of Formula (III) is.

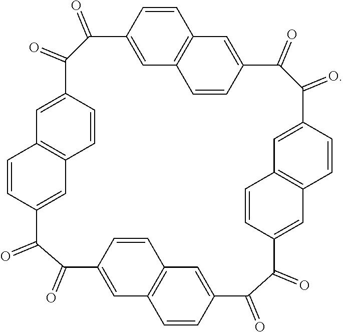 [SMILES: O=C1C(=O)c2ccc3cc(ccc3c2)C(=O)C(=O)c2ccc3cc(ccc3c2)C(=O)C(=O)c2ccc3cc(ccc3c2)C(=O)C(=O)c2ccc3cc1ccc3c2]

In some embodiments, the compound of Formula (V) is:

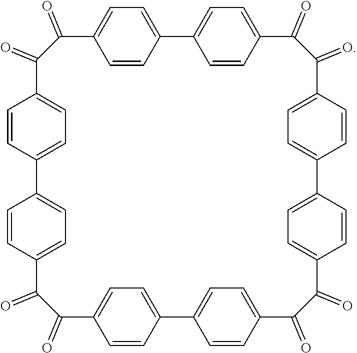 [SMILES: O=C1C(=O)c2ccc(cc2)-c2ccc(cc2)C(=O)C(=O)c2ccc(cc2)-c2ccc(cc2)C(=O)C(=O)c2ccc(cc2)-c2ccc(cc2)C(=O)C(=O)c2ccc(cc2)-c2ccc1cc2]

In some embodiments, the electrode is a cathode electrode. In some embodiments, the cathode electrode is for a battery. In some embodiments, the battery is a lithium-ion battery. In some embodiments, the electrode further comprises at least one of: lithium, a compound comprising lithium, a lithium salt, or a lithium-ion.

In various embodiments, the present invention provides a lithium-ion battery, comprising: an anode electrode, a separator, an electrolyte, and a cathode electrode as described herein. In some embodiments, the electrolyte comprises at least one of: lithium, a compound comprising lithium, a lithium salt, or a lithium-ion.

### BRIEF DESCRIPTION OF THE DRAWINGS

FIG. 1 depicts in accordance with various embodiments of the invention, Cyclotetrabenzil octaketone macrocycle compounds 1-3 (with ring numbering schemes used in the text) and compound 4, the acyclic monomeric counterpart of compound 2.

FIG. 2A-FIG. 2F depicts in accordance with various embodiments of the invention, Charging/discharging profiles of compound 1 (FIG. 2A), compound 2 (FIG. 2B), compound 3 (FIG. 2C) and compound 4 (FIG. 2D). dQ/dV profiles of compound 2 (FIG. 2E) and compound 4 (FIG. 2F) in the electrolyte of LiTFSI in DME/DOL.

FIG. 3A-FIG. 3C depicts in accordance with various embodiments of the invention, Proposed two-step 8e″ reduction for compound 2 (FIG. 3A) and one-step 2e″ reduction for compound 4 (FIG. 3B) Multiplicities (S) are show in parenthesis. FIG. 3C: Computed vertical de″ and 2e″ reduction energies (Ex) for the two-step reduction of compound 2, complex 2-Li₄, complex 2-Li₈ and one-step reduction for compound 4, complex 4-Li₂. Computed distortion energies (ΔE_{dist}), C—C bond lengths, and Wiberg bond indices (WBI) of the carbonyl-linking C—C bonds are also listed.

FIG. 4 depicts in accordance with various embodiments of the invention, Galvanostatic cycling of compound 2 and compound 4 in a 3M LiTFSI in DME/DOL electrolyte. 1C equals 294.5 mA g⁻¹ for compound 2 and 172.7 mA g⁻¹ for compound 4, respectively.

FIG. 5 depicts various embodiments of the invention.

FIG. 6A-FIG. 6B depicts in accordance with various embodiments of the invention, Plotted dQ/dV profiles of compound 1 (FIG. 6A) and compound 3 (FIG. 6B).

FIG. 7A-FIG. 7B depicts in accordance with various embodiments of the invention, Charging/discharging profiles of compound 2 (FIG. 7A) and compound 4 (FIG. 7B) within first 10 cycles.

FIG. 8A-FIG. 8B depicts in accordance with various embodiments of the invention, Charging/discharging profile (FIG. 8A) and normalized capacity-voltage profile (FIG. 8B) of compound 2 in 0.5M and 3M concentrations electrolytes under 0.1 C.

FIG. 9A-FIG. 9F depicts in accordance with various embodiments of the invention, SEM images of the electrode derived from macrocycle compound 2 at three different levels of magnification. (FIG. 9A), (FIG. 9C), and (FIG. 9E) are SEM images of pristine cathode 2 before cycling, while (FIG. 9B), (FIG. 9D), and (FIG. 9F) are SEM images of cathode 2 after 12 cycles under 100 mA g 1 in 0.5 M LiTFSI DME/DOL electrolyte.

FIG. 10A-FIG. 10B depicts in accordance with various embodiments of the invention, SEM images of the electrode derived from macrocycle compound 4 at two different levels of magnification. FIG. 10A is an SEM image of the pristine cathode 4 before cycling, while FIG. 10B is an SEM image of the cathode 4 after 12 cycles under 100 mA g⁻¹ in 0.5 M LiTFSI DME/DOL electrolyte.

FIG. 11A-FIG. 11D depicts in accordance with various embodiments of the invention, Solubility tests for compound 1 (FIG. 11A), compound 2 (FIG. 11B), compound 3 (FIG. 11C), and compound 4 (FIG. 11D), showing the undissolved powders (left) and fully dissolved solution (right) in the 0.5M LiTFSI DME/DOL (v:v=1:1) electrolyte.

FIG. 12 depicts in accordance with various embodiments of the invention, UV-Vis spectra of soaked solutions obtained from compound 2 and compound 4. Inset: separators disassembled from discharging cell and related soak solutions.

FIG. 13 depicts in accordance with various embodiments of the invention, IR Spectra of macrocycle compound 1, macrocycle compound 2, and macrocycle compound 3.

FIG. 14 depicts in accordance with various embodiments of the invention, In situ IR spectra for macrocycle compound 2.

FIG. 15 depicts in accordance with various embodiments of the invention, NICS(0)ᵢₛₒ values at the center of the macrocycle and the six-membered rings for the compound 2, complex 2-Li₄, complex 2-Li₈, compound 4, and complex 4-Li₂ systems computed at the ωB97X-D/def2-SVP level. S stands for multiplicity.

FIG. 16 depicts in accordance with various embodiments of the invention, Relevant CC, CO and OLi distances of compound 2, complex 2-Li₄, complex 2-Li₈, compound 4, and complex 4-Li₂ structures computed at the ωB97X-D/def2-SVP level. S stands for multiplicity.

FIG. 17 depicts in accordance with various embodiments of the invention, Plots of NCI surfaces (s(p)=0.5 isosurface. Color range: −0.035<sign(λ₂)p<+0.02 a.u.) for compound 2, complex 2-Li₄, complex 2-Li₈, compound 4, complex 4-Li₂. The red colored surfaces indicate repulsive interactions, the green colored indicate van der Waals interactions, and the blue colored ones indicate attractive interactions.

FIG. 18A-FIG. 18B depicts in accordance with various embodiments of the invention, FIG. 18A: Proposed two-step 8e⁻ reduction for compound 1. Multiplicities (S) are shown in parenthesis; FIG. 18B: Computed vertical 4e⁻ reduction energies (Eᵥ) for the two-step reduction of compound 1. Computed distortion energies (ΔE_{dist}), C—C bond lengths, and Wiberg bond indices (WBI) of the carbonyl-linking C—C bonds also are listed.

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, apparatus, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

In some embodiments, the numbers expressing quantities of reagents, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

As used herein the term “electron donating group” is well-known in the art and generally refers to a functional group or atom that pushes electron density away from itself, towards other portions of the molecule, e.g., through resonance and/or inductive effects. Non-limiting examples of electron-donating groups include OR^{c}, NR^{c}R^{d}, alkyl groups, wherein R^{c} and R^{d} are each independently H, optionally substituted alkyl, optionally substituted heteroalkyl, optionally substituted aryl, optionally substituted heteroaryl, optionally substituted cyclyl, or optionally substituted heterocyclyl.

As used herein the term “electron withdrawing group” is well-known in the art and generally refers to a functional group or atom that pulls electron density towards itself, away from other portions of the molecule, e.g., through resonance and/or inductive effects. Non-limiting examples of electron withdrawing groups include NO₂, F, Cl, Br, I, CF₃, CN, CO₂R^{a}, C(═O)NR^{a}R^{b}, C(═O)R^{a}, SO₂R^{a}, SO₂OR^{a}, SO₂NR^{a}R^{b}, PO₃R^{a}R^{b}, or NO, wherein R^{a} and R^{b} are each independently H, optionally substituted alkyl, optionally substituted heteroalkyl, optionally substituted aryl, optionally substituted heteroaryl, optionally substituted cyclyl, or optionally substituted heterocyclyl.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In preferred embodiments, a straight chain or branched chain alkyl has 30 or fewer carbon atoms in its backbone (e.g., C1-C30 for straight chains, C3-C30 for branched chains), and more preferably 20 or fewer. Likewise, preferred cycloalkyls have from 3-10 carbon atoms in their ring structure, and more preferably have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Throughout the application, preferred alkyl groups are lower alkyls. In preferred embodiments, a substituent designated herein as alkyl is a lower alkyl.

Non-limiting examples of substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 2 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkelyne, and alkynylene” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b} Non-limiting examples of Rₐ and R_{b} are each independently hydrogen, alkyl, substituted alkyl, alkenyl, or substituted alkenyl. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine (F), chlorine (Cl), bromine (Br) and iodine (I). The term “halogen radioisotope” or “halo radioisotope” refers to a radionuclide of an atom selected from fluorine (F), chlorine (Cl), bromine (Br) and iodine (I).

As used herein the term “iodo” refers to the iodine atom (I) when it is used in the context of a halo functional group or halogen functional group or as a halo substituent or halogen substituent.

As used herein the term “bromo” refers to the bromine atom (Br) when it is used in the context of a halo functional group or halogen functional group or as a halo substituent or halogen substituent.

As used herein the term “chloro” refers to the chlorine atom (Cl) when it is used in the context of a halo functional group or halogen functional group or as a halo substituent or halogen substituent.

As used herein the term “fluoro” refers to the fluorine atom (F) when it is used in the context of a halo functional group or halogen functional group or as a halo substituent or halogen substituent.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. For example, C₆-C₁₂ aryl includes aryls that have 6 to 12 carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. For example, C₄-C₉ heteroaryl includes heteroaryls that have 4 to 9 carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2(1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cycyl are typically used where X and Y indicate the number of carbon atoms in the ring system. For example, C₃-C₈ cyclyl includes cyclyls that have 3 to 8 carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, —CN, or the like.

The term “heterocyclyl” refers to a nonaromatic 4-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. For example, C₄-C₉ heterocyclyl includes heterocyclyls that have 4-9 carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring.

Substituents may be protected as necessary and any of the protecting groups commonly used in the art may be employed. Non-limiting examples of protecting groups may be found, for example, in Greene et al., Protective Groups in Organic Synthesis, 3rd Ed. (New York: Wiley, 1999).

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In preferred embodiments, the “alkylthio” moiety is represented by one of —S— alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), —N(C₁-C₁₀alkyl)₂, and the like. The term “alkylamino” includes “alkenylamino.” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example —NHheteroaryl, and —N(heteroaryl)₂. Optionally, two substituents together with the nitrogen can also form a ring Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino” —NH-(alkyl)-(cycloalkyl), such as —NHCH₂-cyclohexyl, and the like.

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and Re can each independently be hydrogen or any other substituent where the atom alpha to the carbon is a heteroatom or cyano. Hence, CF₃, CH₂OH and CH₂CN are all C₁ alkyls.

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

In various embodiments, compounds of the present invention as disclosed herein may be synthesized using any synthetic method available to one of skill in the art. Non-limiting examples of synthetic methods used to prepare various embodiments of compounds of the present invention are disclosed in the Examples section herein.

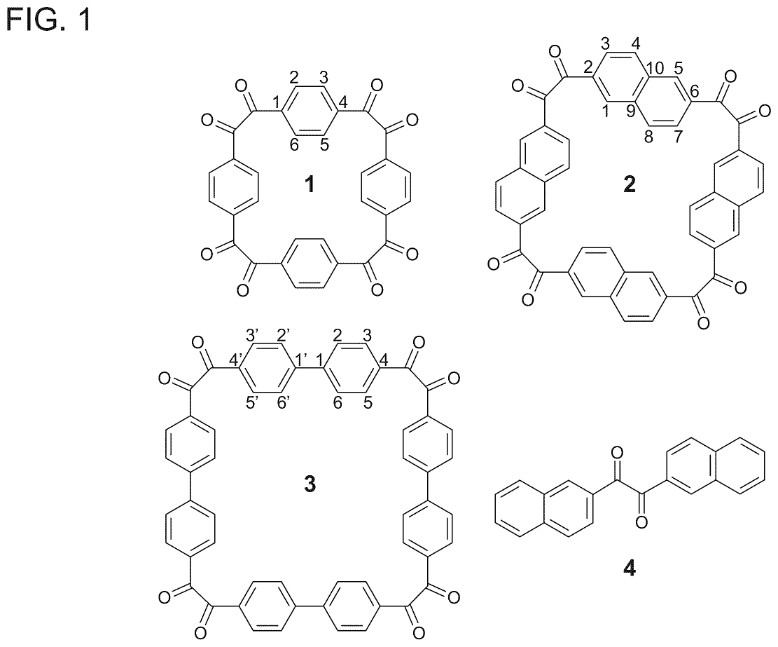

Organic electrode materials could revolutionize batteries because of their high energy densities, the use of Earth-abundant elements, and structural diversity, which allows fine-tuning of electrochemical properties. However, small organic molecules and intermediates formed during their redox cycling in lithium-ion batteries (LIBs) have high solubility in organic electrolytes, leading to rapid decay of cycling performance. Herein we report the use of three cyclotetrabenzil octaketone macrocycles, compounds 1-3 (FIG. 1), as cathode materials for lithium-ion batteries (LIBs) The rigid and insoluble naphthalene-based cyclotetrabenzil reversibly accepts eight electrons in a two-step process with a specific capacity of 279 mAh g⁻¹ and a stable cycling performance with ˜65% capacity retention after 135 cycles. DFT calculations indicate that its reduction increases both ring strain and ring rigidity, as demonstrated by computed high distortion energies, repulsive regions in NCI plots, and close [C . . . C] contacts between the naphthalenes. This work highlights the importance of shape-persistency and ring strain in the design of redox-active macrocycles that maintain very low solubility in various redox states.

Naphthalene-based cyclotetrabenzil octaketone compound 2 is an excellent cathode material for lithium-ion batteries. It reversibly accepts eight electrons per molecule, has a specific capacity of 279 mAh g⁻¹, and shows stable cycling performance with ˜65% retention after 135 cycles. Its shape-persistent, symmetric structure rigidifies during an eight-electron reduction, ensuring that this macrocycle remains insoluble in various redox states.

Herein we report the synthesis and electrochemical investigations of tetrameric cyclotetrabenzil-based macrocycle compounds 1-3 with eight carbonyl functional groups (FIG. 1), as well as compound 4: the acyclic counterpart of compound 2 with only two carbonyl groups. The electrochemical results show that, despite structural similarity, compounds 1-3 behave very differently when included into organic battery electrodes. The naphthalene-based macrocycle compound 2 stands out as the only one to reversibly store up to eight electrons. The key to its superior performance is the shape-persistent structure, which ensures low solubility in the electrolyte throughout the redox cycling. This study offers important insights into the potential of shape-persistent, redox-active macrocyclic compounds as motifs for the construction of organic electrode materials.

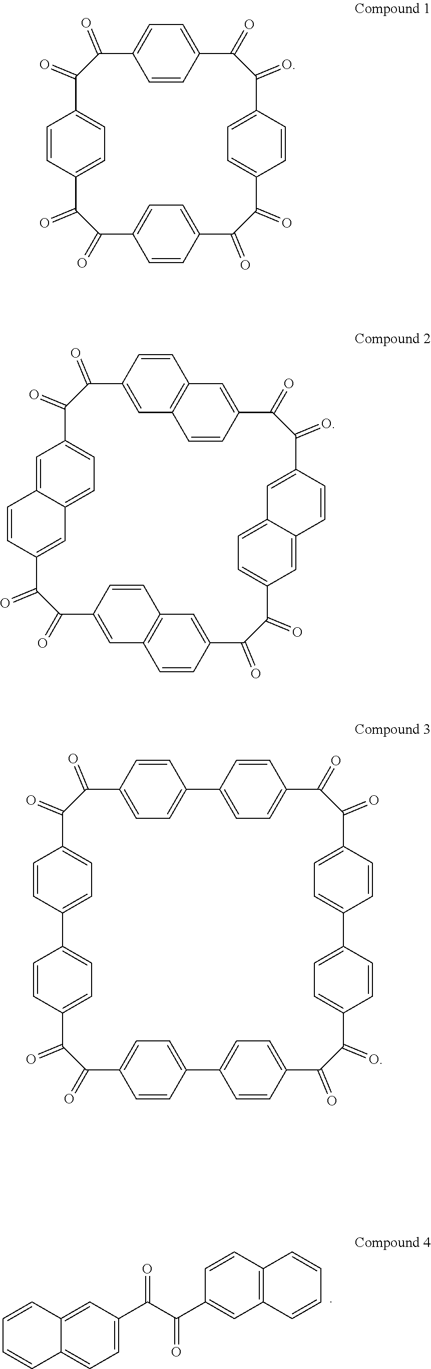

Macrocycle compound 1 has been reported in the literature. Compound 2 and compound 3 were prepared by the oxidation of expanded cyclotetrabenzoins as described herein. The acyclic monomeric 1,2-bis(2-naphthyl)ethan-1,2-dione (compound 4) was prepared by the oxidation of the corresponding benzoin, which was in turn produced by the benzoin condensation of 2-formylnaphthalene as described herein. Compounds 1-4 are air-stable and contain either one or four pairs of redox-active carbonyl groups. Their spectroscopic characterization data are consistent with their symmetry; for all compounds, a single ¹³C NMR signal for their carbonyl groups is observed, indicating their equivalence. From the crystal structures of analogs of compounds 1-3, the central cavities with approximate dimensions of 6.8×6.8 Å (compound 1), 8.6×8.6 Å (compound 2), and 10.1×11.9 Å (compound 3). Details of syntheses and spectroscopic properties of compounds 2-4 are given herein.

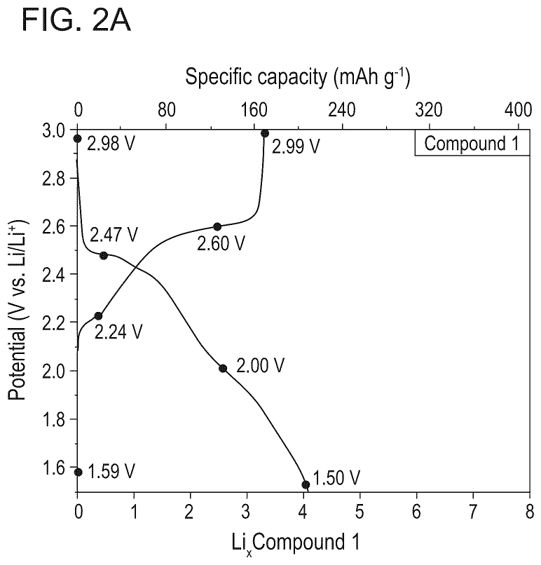

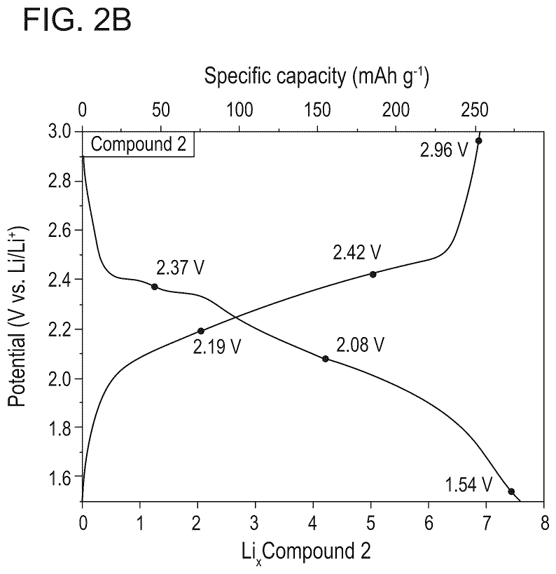

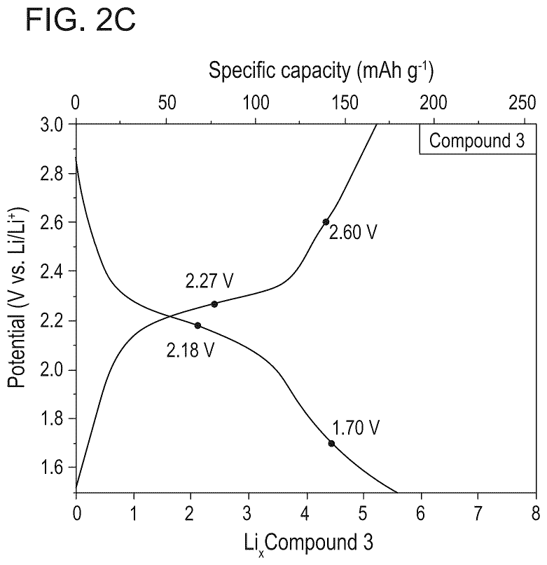

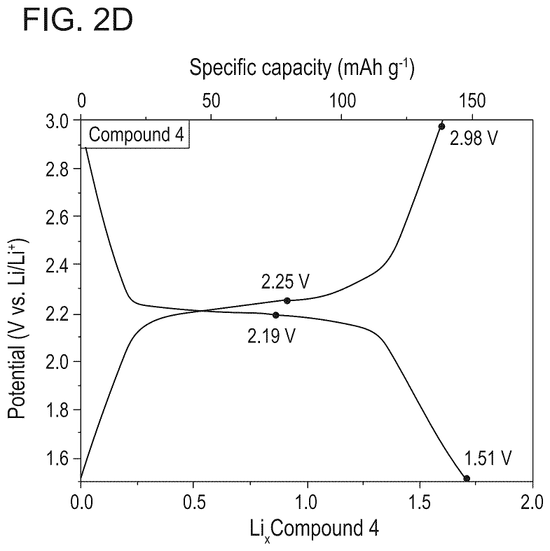

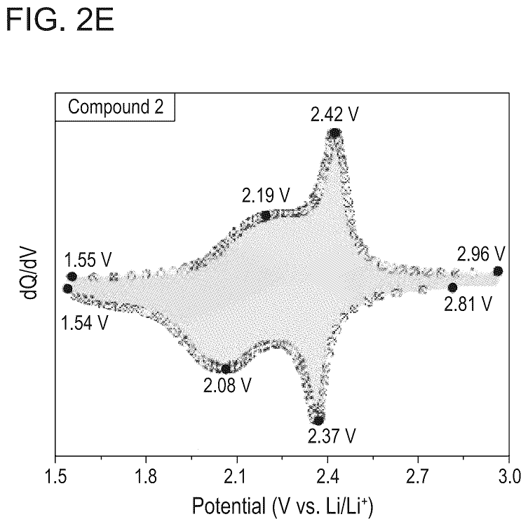

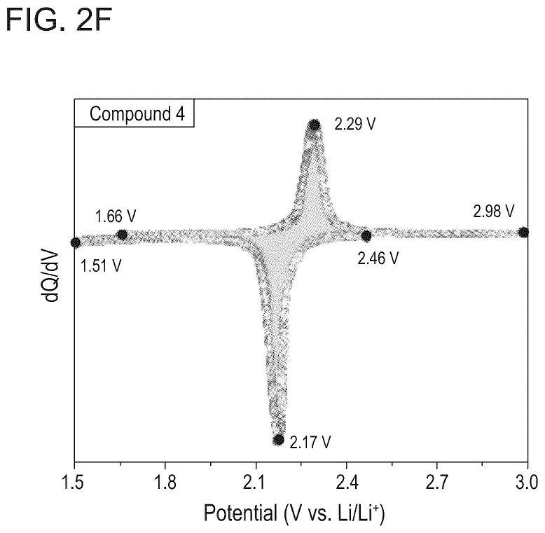

With compounds 1-4 in hand, we evaluated their electrochemical redox behavior as the active components within the organic electrodes. FIG. 2A-FIG. 2D show the charge/discharge profiles during typical cycling for compounds 1-4 under the current density of 100 mA g⁻¹, and using lithium bis-trifluoromethanesulfonimide (LiTFSI) in dioxolane/dimethoxyethane (DOL/DME) as the electrolyte. Assuming the transfer of eight electrons to their carbonyl groups, the theoretical specific capacities of compounds 1-3 are 406.02, 294.46, and 257.44 mAh g⁻¹, respectively. However, compound 1 showed a notably lower specific capacity of 171 mAh g⁻¹. Compound 3 displayed an irreversible discharging tail near the cut-off voltage. In contrast, the discharge capacity for compound 2 in a typical cycle was found to be 279 mAh g⁻¹ in a 3M electrolyte solution (FIG. 2B), which is ˜95% of the theoretical capacity. This capacity confirmed that a molecule of compound 2 can reversibly store up to eight electrons during the electrochemical cycling; compound 1 could store only up to four electrons. Similarly, compound 4 with two carbonyl groups exhibited the discharging capacity of 148.7 mAh g⁻¹ (FIG. 2D), which also approached its predicted specific capacity of 172.7 mAh g⁻¹, confirming that all the carbonyl groups within the molecule are electrochemically active.

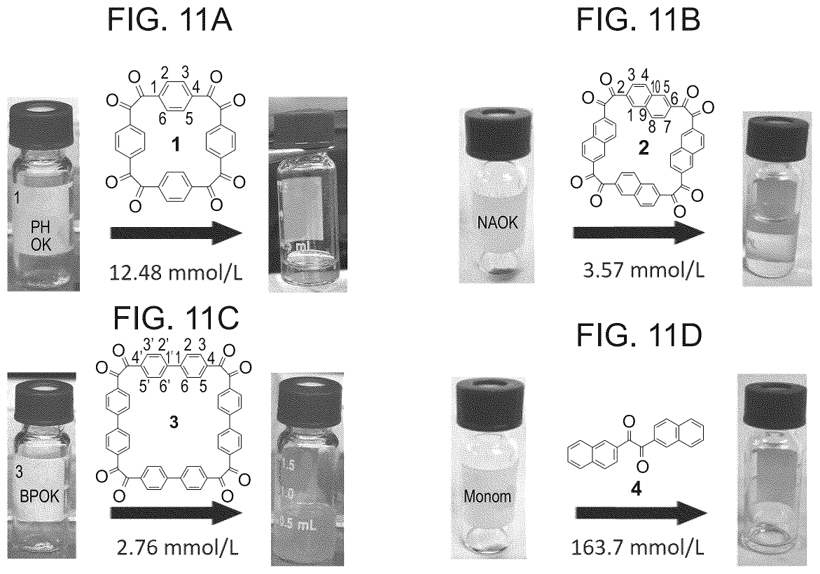

We ascribed the poorer performance of compound 3 to its higher solubility in the electrolyte, either in their neutral and/or partially reduced states. Macrocycle compound 3 has a large cavity space and thus free rotation of the biphenyl linkers can increase solubility, as suggested by a low computed rotational barrier (1.7 kcal mol⁻¹, cf. 12 kcal mol⁻¹ for 2 and 11.4 kcal mol⁻¹ for 1). The reasons for the inferior performance of compound 1 are discussed below in the computational section. We therefore focused our further investigation on the best-performing macrocycle compound 2, as well as its model compound 4. The solubility of compound 2 in the 0.5M solution of LiTFSI in DME/DOL electrolyte is 3.6±0.1 mmol L⁻¹ (FIG. 11B), while that of compound 4 is 164±1 mmol L⁻¹ (FIG. 11D). The much lower solubility of compound 2 is tentatively ascribed to its symmetric structure which facilitates crystal packing and the free rotation of naphthalene moieties in the acyclic compound 4.

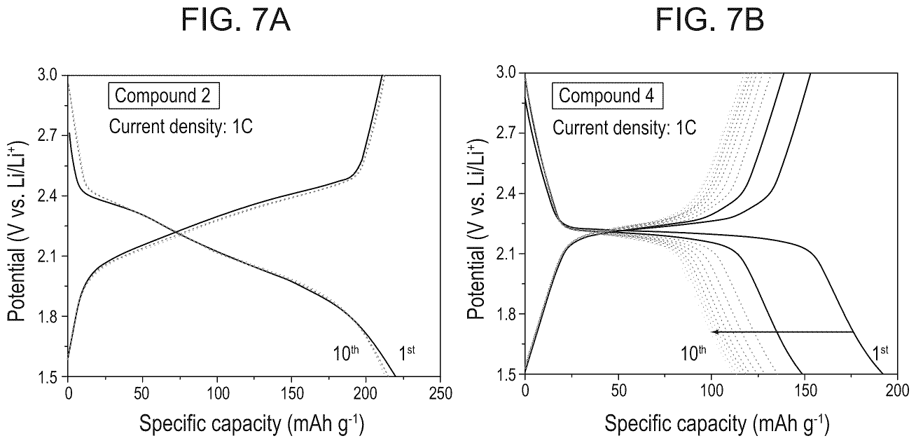

For these two compounds, dQ/dV vs. potential dependencies are plotted in FIG. 2E-FIG. 2F and FIG. 7A-FIG. 7B based on the charging/discharging profiles. Two pairs of redox peaks are observed for compound 2 (FIG. 2E) at 2.37 and 2.08 V (for lithiation), suggesting that, without being bound by theory, eight electrons are transferred in two separate steps; different shapes of these two pairs of peaks also indicate different electron transfer kinetics. In contrast, compound 4 only shows one pair of peaks in its dQ/dV plot (2.17 V for lithiation, FIG. 2F), indicating that it achieves all its capacity in an apparent one-step reaction.

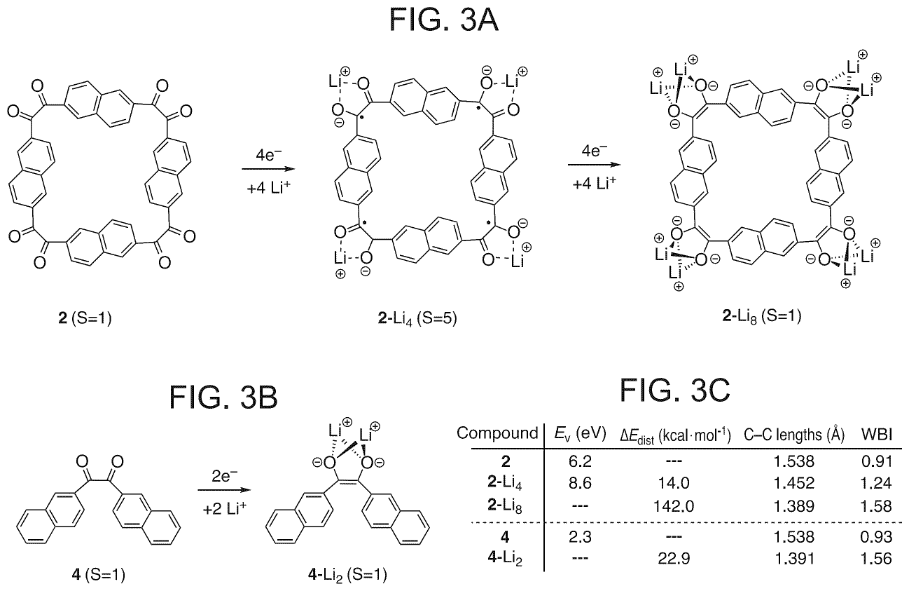

To better understand the difference in the reaction mechanism between compound 2 and compound 4, we performed computational studies. DFT calculations for compound 2 and compound 4 and their reduced, Lit-bound complexes were performed to provide insights into structural factors that influence the reduction behavior of the macrocycle versus its truncated acyclic model. Macrocycle compound 2 undergoes a two-step eight-electron reduction process (FIG. 3A).

In the first step, four electrons add to compound 2 and four Li⁺ ions bind to the four pairs of neighboring carbonyl groups, forming complex 2-Li₄. In the second step, four more electrons are added to complex 2-Li₄ and four more Lit ions bind to the four pairs of neighboring partially reduced carbonyl groups, forming a near-tetrahedral arrangement between the two Li and two O atoms in complex 2-Li₈. In agreement with the experiment, vertical attachment of four electrons onto compound 2 is endothermic (Eᵥ=6.2 eV. 2→2⁴⁻), but the addition of the next four electrons to complex 2-Lis is even more so (Eᵥ=8.6 eV, 2-Li₄→2-Li⁴⁻; FIG. 3C).

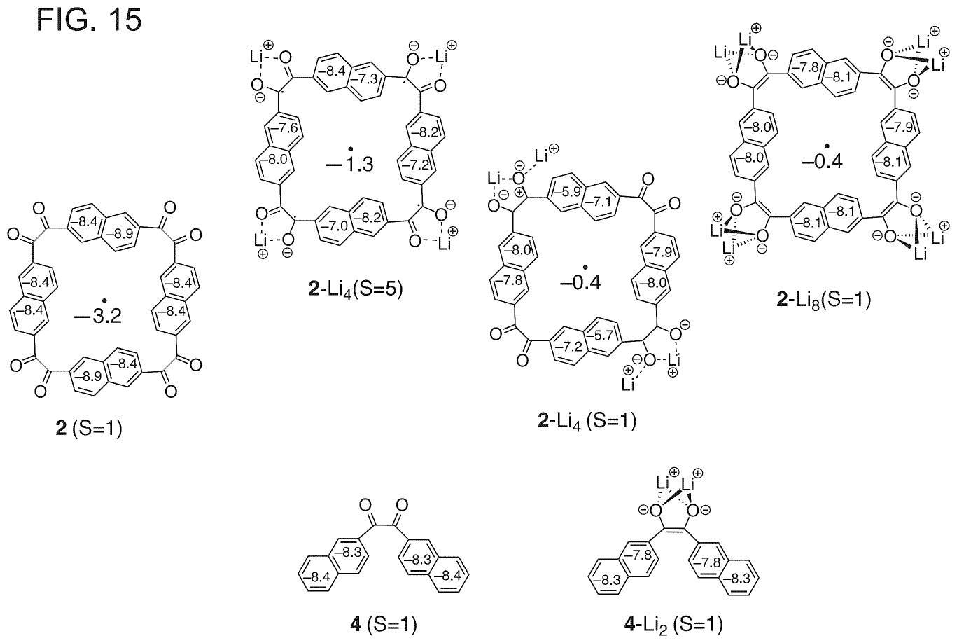

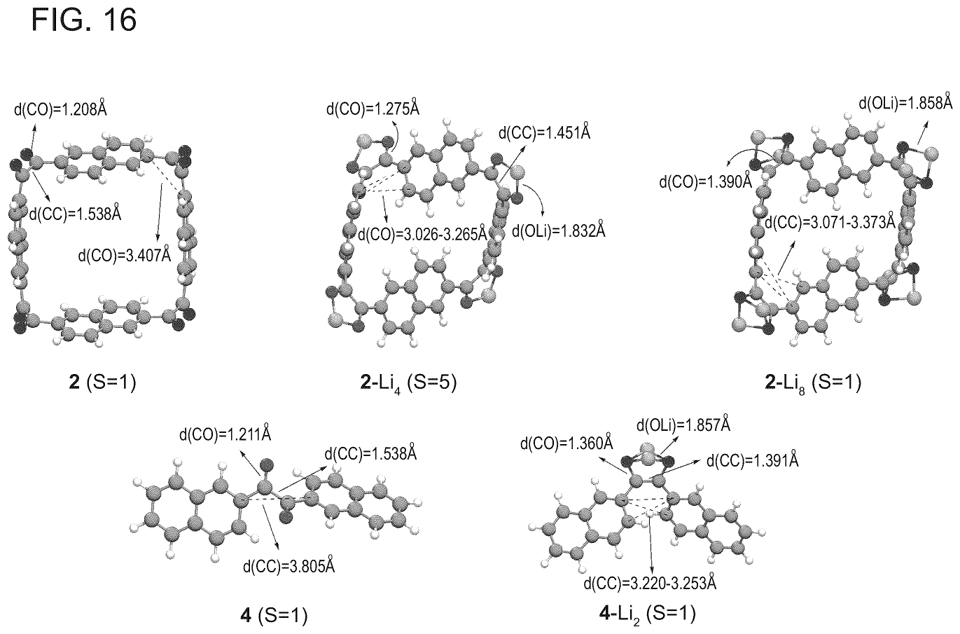

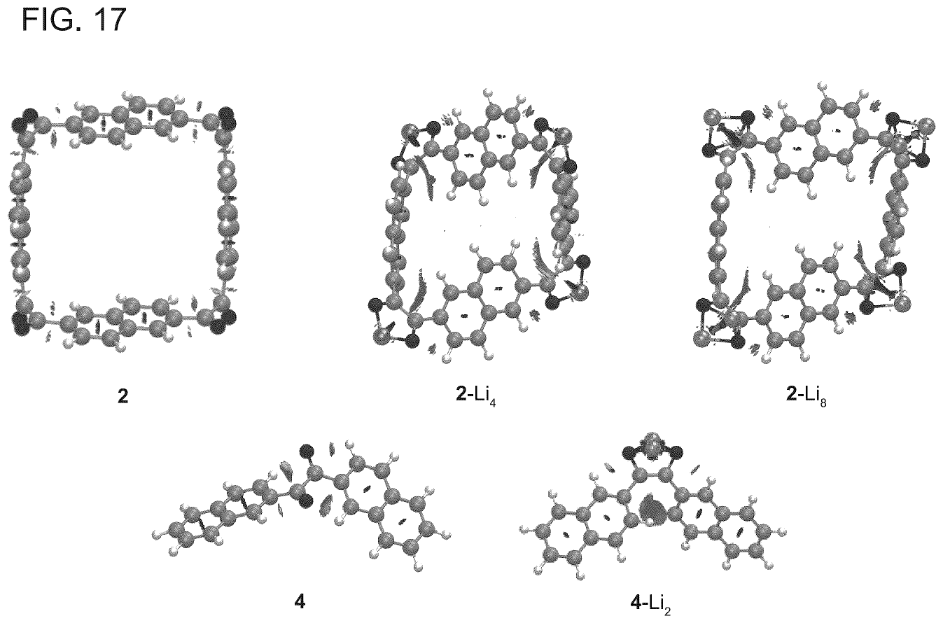

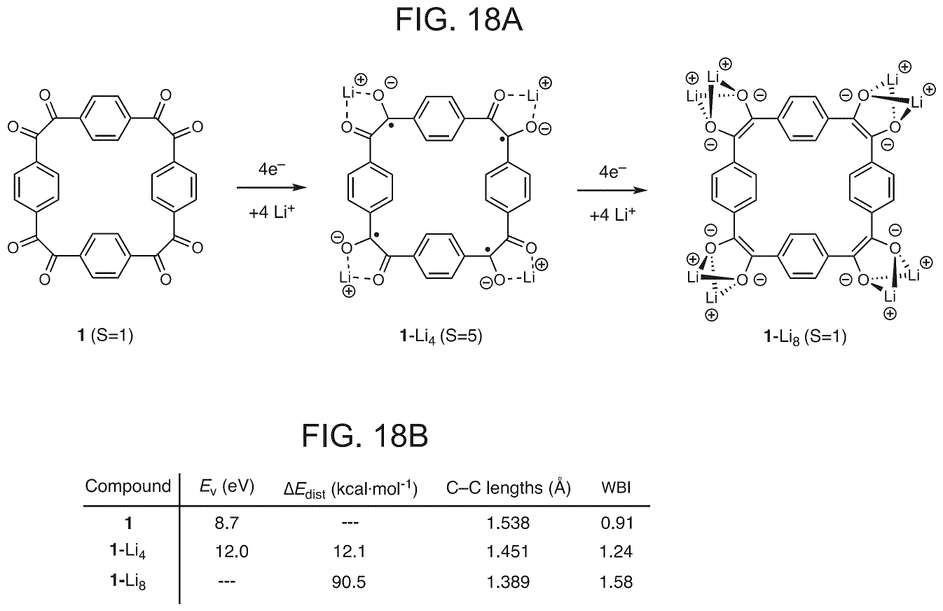

Computations show that reduction of compound 2 is localized at the four dicarbonyl sites. Notably, aromaticity of the naphthalene linkers remains intact throughout the reduction process (see computed nucleus independent chemical shifts, NICS, results in FIG. 15) In compound 2, all of the carbonyl-linking C—C bonds are formal single bonds (C—C=1.538 Å, Wiberg bond index, WBI=0.91, FIG. 3C). Upon four-fold reduction to complex 2-Li₄, four of the carbonyl groups (C═O) are converted to ketyl radical anions (·C—O⁻), and the resulting allylic conjugation shortens the carbonyl-linking C—C bonds (C—C=1.452 Å, WBI=1.24). Complex 2-Li₄ is a quintuplet ground state with spins localized on the carbonyl sites. In complex 2-Li₈, the addition of the next four electrons reduces all of the carbonyl groups, forming eight C—O⁻ anions, and the carbonyl-linking C—C bonds shorten even further, gaining pronounced π-bond character (C—C=1.389 Å and WBI=1.58). Eight electron reduction introduces a notable amount of strain into the macrocycle. In complex 2-Li₈, the edge C—C bonds of each neighboring naphthalene linker are within the combined van der Waals radii of two C's (close contact [C . . . C] distances ranging from 3.071 to 3.373 Å, see geometries in the FIG. 16), and increased rigidity of the carbonyl-linking C—C bonds precludes possible conformational changes that may relieve this repulsive interaction. The computed distortion energy (ΔE_{dist}) for complex 2-Li₄ is 14.0 kcal mol⁻¹, while that for complex 2-Li₈ is 142.0 kcal mol⁻¹, showing significantly greater energetic penalty for reorienting the naphthalene linkers in complex 2-Lis (FIG. 3C). Values for ΔE_{dist} were estimated based on the single point total electronic energies of complex 2-Li₄ and complex 2-Li₈ with Li⁺ ions removed minus the total electronic energies of 24- and 28-, respectively. Computed noncovalent interaction (NCI) plots for compound 2, complex 2-Li₄, and complex 2-Li₈ are given herein in FIG. 17. Even though macrocycle compound 1 is structurally similar to compound 2, the energetic penalty for 4e⁻ reduction is noticeably higher (Eᵥ=8.7 eV for 1→1⁴⁻ and 12.0 eV for 1-Li₄→1-Li₄⁴⁻, cf. data in FIG. 3C), possibly due to a smaller cavity space.

Acyclic compound 4 is a truncated fragment of compound 2 and undergoes a one-step two-electron reduction (FIG. 3B). In compound 4, the carbonyl groups form a 123.2° O—C—C—O dihedral angle. Upon twofold reduction, Li⁺ ions bind to the two carbonyl groups forming a near-tetrahedral arrangement between the two Li and two O atoms. The reduced carbonyl groups are now in a cisoid conformation with a 6.2° O—C—C—O dihedral angle. Notably, vertical 2e⁻ attachment to compound 4 is less endothermic (Eᵥ=2.3 eV, 4→4²⁻) than half of the vertical 4e⁻ attachment of compound 2 (½ Eᵥ=3.1 eV, FIG. 3C). The carbonyl-linking C—C bond is a formal single bond in compound 4 (C—C=1.538 Å, WBI=0.93) but shortens significantly in complex 4-Li₂ (C—C=1.391 Å, WBI=1.56). In complex 4-Li₂, the edge C—C bonds of each neighboring naphthalene linkers are within the combined van der Waals radii of two C's (close contact [C . . . C] distances ranging from 3.220 to 3.253 Å, see geometries in the FIG. 16). However, the estimated distortion energy for complex 4-Li₂ (ΔE_{dist}=22.9 kcal mol⁻¹) is less than a quarter of the distortion energy of complex 2-Li₈ (¼ ΔE_{dist}=35.5 kcal mol⁻¹) These results explain the observed two-step reduction of macrocycle compound 2. The addition of the first four electrons creates ketyl radical anions, which retain most of the conformational flexibility of the neutral compound 2. Subsequent reductions are more difficult as they create strained and rigid dianions. Without being bound by theory, we speculate that this progressive rigidification in the 2→2-Li₄→2-Li₈ series also results in desirable lowered solubility of the reduced species relative to compound 2.

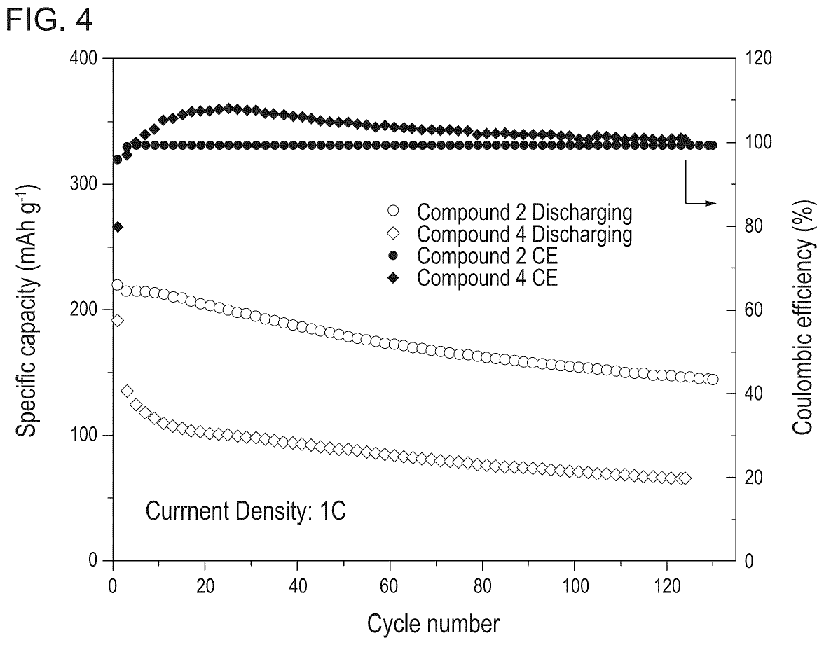

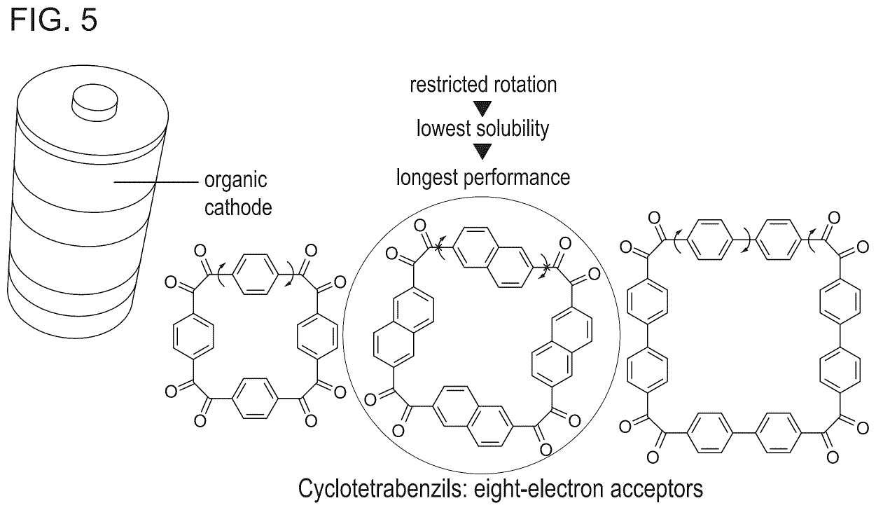

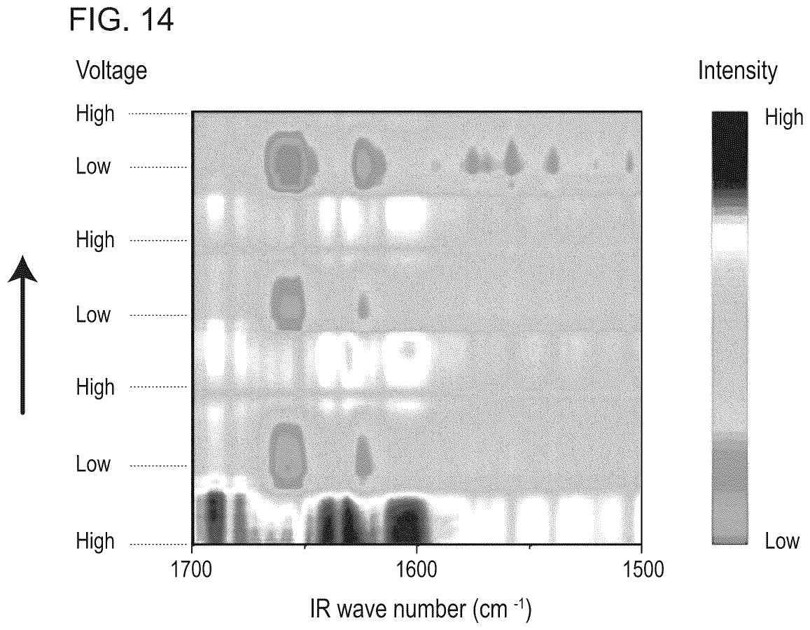

FIG. 4 demonstrates the galvanostatic cycling of compound 2 and compound 4 in 3M LiTFSI electrolyte at 1C. Cathodes made with macrocycle compound 2 exhibit stable cycling with columbic efficiency ˜99%. After 135 cycles, the discharge capacity retention of compound 2 is 65%. It can be more clearly seen from FIG. 7B that the capacity of compound 4 decays quickly due to its faster dissolution even in the 3M electrolyte. In situ infrared spectra of compound 2 (FIG. 14) show the C═O signal at wavenumbers between 1700 and 1600 cm⁻¹ disappearing during discharging and reappearing during charging, indicating good cycling reversibility of compound 2 as the organic cathode for lithium-ion storage.

In conclusion, we have compared three cyclotetrabenzil octaketone macrocycles as organic cathode materials for lithium-ion storage. Among them, macrocycle compound 2 exhibits a desirable balance of electronic and rigid structural properties that support efficient electrode reactions. Compound 2 has a specific capacity of 279 mAh g⁻¹ from a reversible eight-electron reduction, and displays a good cycling performance with 65% of the initial capacity retained after 135 cycles. The naphthalene-based cyclotetrabenzil octaketone compound 2 reported herein demonstrated a low-capacity decay at a current density of 1C, while maintaining a high-capacity higher than 150 mAh g⁻¹ over 120 cycles. Investigation of the fundamental electron transfer processes for compound 2 and compound 4 revealed that compound 2 undergoes an eight-electron reduction in two discrete steps, while compound 4 achieves full reduction in a single step. Without being bound by theory, computational studies suggest that macrocycle compound 2 experiences moderate macrocyclic strain in the first step of the reduction process, but significant strain in the second step. These results highlight the importance of modulating macrocyclic strain in the design of organic cathode materials. This study provides new insights into the role of ring strain, noncovalent interactions, and multielectron transfer energies of organic electrode material design, and offers a kinetic understanding for well-designed macrocycles in electrochemical process.

### Various Embodiments of the Invention

### Embodiments Include Those Listed Below

Embodiment 1. A compound of Formula (I):

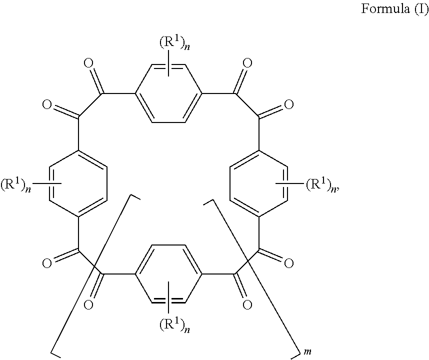

wherein:
m is 0, 1, 2, 3, or 4;
n is 0, 1, 2, 3 or 4; and
R¹ is an electron withdrawing group, or an electron donating group.

Embodiment 2. A compound of embodiment 1, wherein the compound is a compound of Formula (I-a):

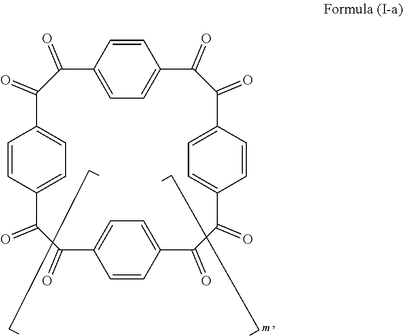

wherein:
m is 0, 1, 2, 3, or 4.

Embodiment 3. A compound of embodiment 1, wherein the compound is a compound of Formula (I-b):

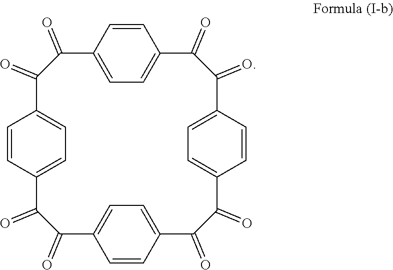 [SMILES: O=C1C(=O)c2ccc(cc2)C(=O)C(=O)c2ccc(cc2)C(=O)C(=O)c2ccc(cc2)C(=O)C(=O)c2ccc1cc2]

Embodiment 4. A compound of Formula (II):

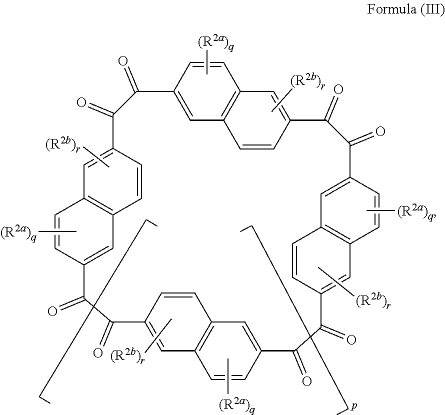

wherein:
p is 0, 1, 2, 3, or 4;
q is 0, 1, 2, or 3;
r is 0, 1, 2, or 3;
R^{2a} is an electron withdrawing group, or an electron donating group; and
R^{2b} is an electron withdrawing group, or an electron donating group.

Embodiment 5. A compound of embodiment 4, wherein the compound is a compound of Formula (III-a):

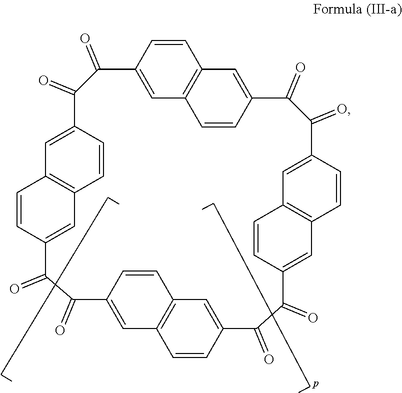

wherein:
p is 0, 1, 2, 3, or 4.

Embodiment 6. A compound of embodiment 4, wherein the compound is a compound of Formula (III-b):

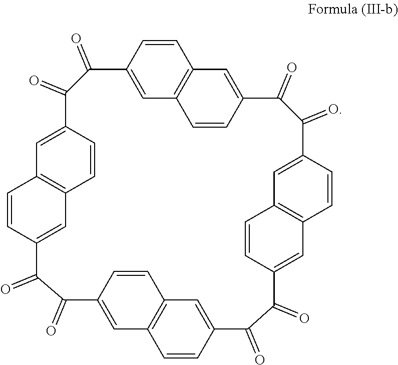 [SMILES: O=C1C(=O)c2ccc3cc(ccc3c2)C(=O)C(=O)c2ccc3cc(ccc3c2)C(=O)C(=O)c2ccc3cc(ccc3c2)C(=O)C(=O)c2ccc3cc1ccc3c2]

Embodiment 7. A compound of Formula (V):

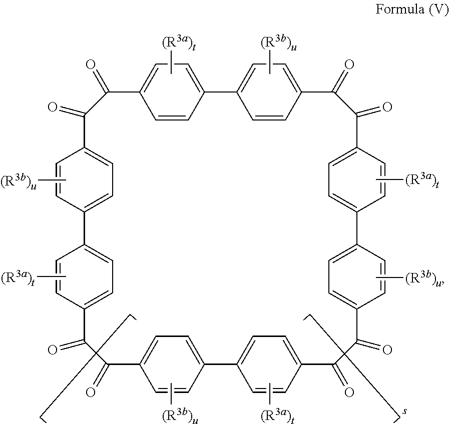

wherein:
s is 0, 1, 2, 3, or 4;
t is 0, 1, 2, 3 or 4;
u is 0, 1, 2, 3, or 4;
R^{3a} is an electron withdrawing group, or an electron donating group; and
R^{3b} is an electron withdrawing group, or an electron donating group.

Embodiment 8. A compound of Formula (V-a):

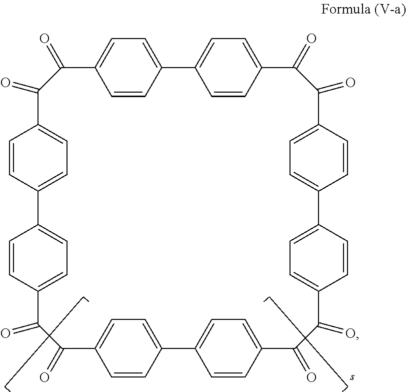

wherein:
s is 0, 1, 2, 3, or 4.

Embodiment 9. A compound of Formula (V-b):

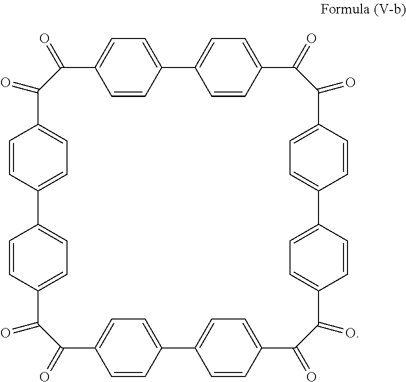 [SMILES: O=C1C(=O)c2ccc(cc2)-c2ccc(cc2)C(=O)C(=O)c2ccc(cc2)-c2ccc(cc2)C(=O)C(=O)c2ccc(cc2)-c2ccc(cc2)C(=O)C(=O)c2ccc(cc2)-c2ccc1cc2]

In some embodiments, a compound of Formula (I-a) is a compound of Formula (I). In some embodiments, a compound of Formula (I-b) is a compound of Formula (I). In some embodiments, a compound of Formula (I-b) is a compound of Formula (I-a).

In some embodiments, a compound of Formula (III-a) is a compound of Formula (III). In some embodiments, a compound of Formula (III-b) is a compound of Formula (III). In some embodiments, a compound of Formula (III-b) is a compound of Formula (III-a).

In some embodiments, a compound of Formula (V-a) is a compound of Formula (V). In some embodiments, a compound of Formula (V-b) is a compound of Formula (V). In some embodiments, a compound of Formula (V-b) is a compound of Formula (V-a).

Additional embodiments include those listed below:

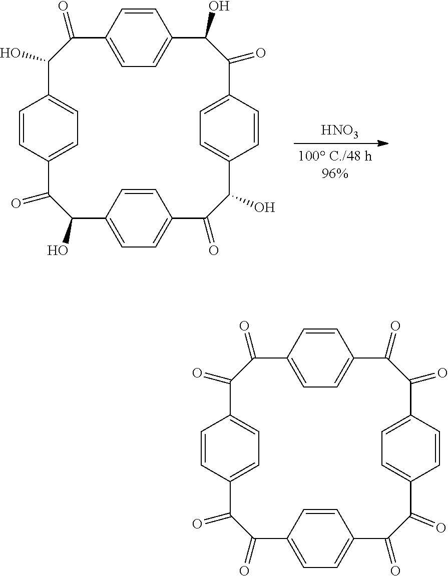

Additional embodiments include those listed below:

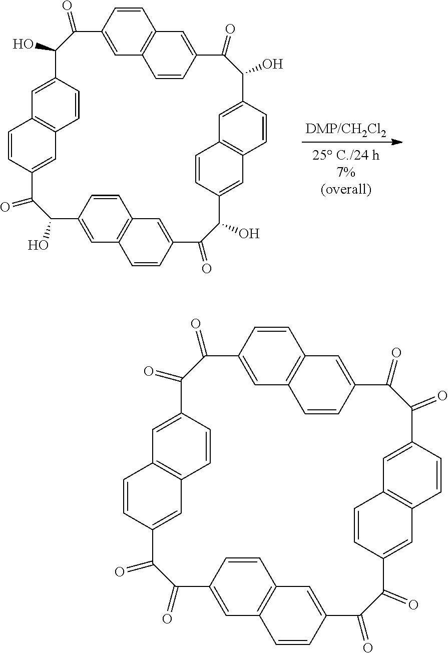 [SMILES: O=C1c2ccc3cc(ccc3c2)[C@@H](O)C(=O)c2ccc3cc(ccc3c2)C(=O)[C@H](O)c2ccc3cc(ccc3c2)[C@H](O)C(=O)c2ccc3cc(ccc3c2)[C@@H]1O ; O=C1C(=O)c2ccc3cc(ccc3c2)C(=O)C(=O)c2ccc3cc(ccc3c2)C(=O)C(=O)c2ccc3cc(ccc3c2)C(=O)C(=O)c2ccc3cc1ccc3c2]

Additional embodiments include those listed below:

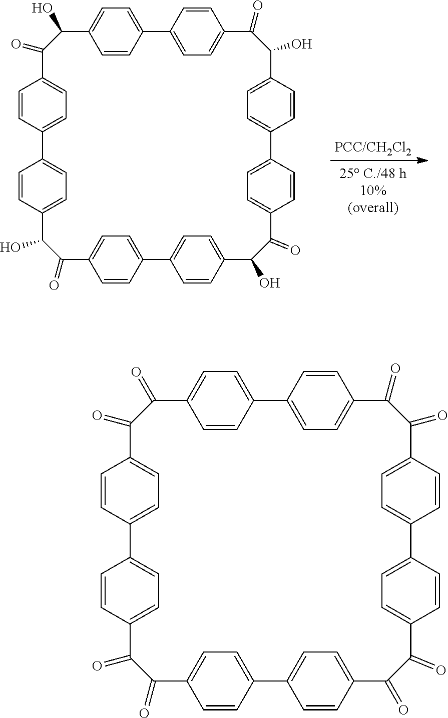 [SMILES: O=C1c2ccc(cc2)-c2ccc(cc2)[C@@H](O)C(=O)c2ccc(cc2)-c2ccc(cc2)[C@H](O)C(=O)c2ccc(cc2)-c2ccc(cc2)[C@@H](O)C(=O)c2ccc(cc2)-c2ccc(cc2)[C@@H]1O ; O=C1C(=O)c2ccc(cc2)-c2ccc(cc2)C(=O)C(=O)c2ccc(cc2)-c2ccc(cc2)C(=O)C(=O)c2ccc(cc2)-c2ccc(cc2)C(=O)C(=O)c2ccc(cc2)-c2ccc1cc2]

Additional embodiments include those listed below:

In various embodiments, the present invention provides a complex having a structure:

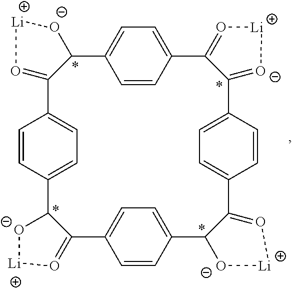

In various embodiments, the present invention provides a complex having a structure:

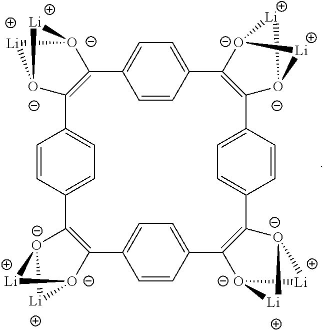

In various embodiments, the present invention provides a complex having a structure:

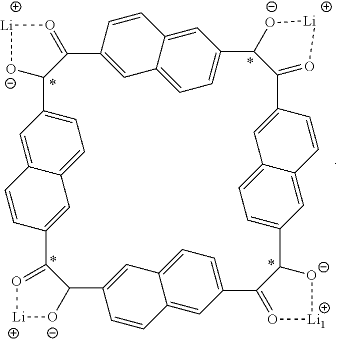

In various embodiments, the present invention provides a complex having a structure:

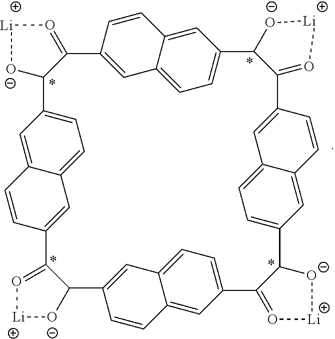

In various embodiments, the present invention provides a complex having a structure:

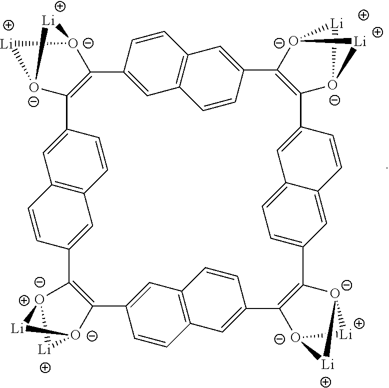

Additional embodiments include those listed below:

Embodiment 1A. An electrode, comprising at least one compound, wherein the at least one compound is selected from the group consisting of a compound of Formula (I), a compound of Formula (III), and a compound of Formula (V), and any combinations thereof; wherein the compound of Formula (I) is:

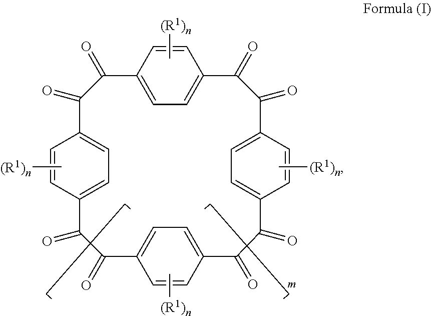

wherein:
m is 0, 1, 2, 3, or 4;
n is 0, 1, 2, 3 or 4; and
R¹ is an electron withdrawing group, or an electron donating group;
   wherein the compound of Formula (III) is:

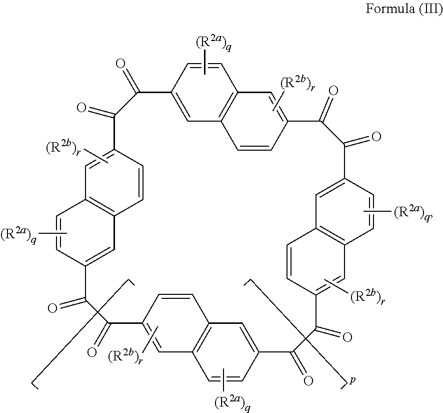

wherein:
p is 0, 1, 2, 3, or 4;
q is 0, 1, 2, or 3;
r is 0, 1, 2, or 3;
R^{2a} is an electron withdrawing group, or an electron donating group; and
R^{2b} is an electron withdrawing group, or an electron donating group; and
   wherein the compound of Formula (V) is:

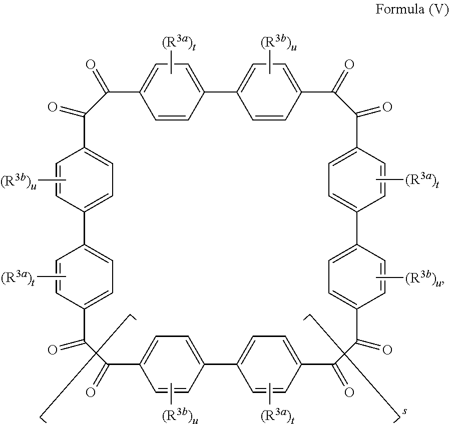

wherein:
s is 0, 1, 2, 3, or 4;
t is 0, 1, 2, 3 or 4;
u is 0, 1, 2, 3, or 4;
R^{3a} is an electron withdrawing group, or an electron donating group; and
R^{3b} is an electron withdrawing group, or an electron donating group.

Embodiment 2A. The electrode of embodiment 1A, wherein the compound of Formula (I) is a compound of Formula (I-a):

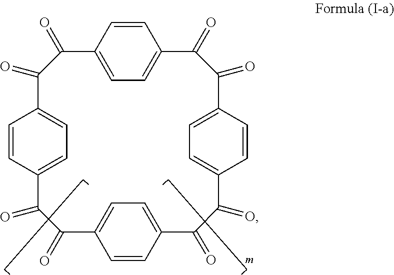

wherein:
m is 0, 1, 2, 3, or 4;
   wherein the compound of Formula (III) is a compound of Formula (III-a):

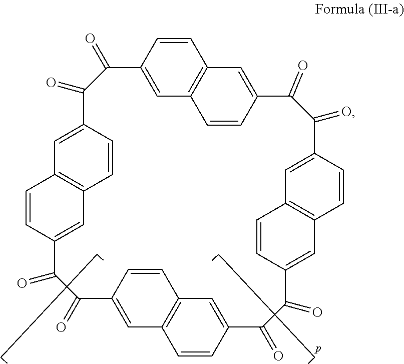

wherein:
p is 0, 1, 2, 3, or 4; and
   wherein the compound of Formula (V) is a compound of Formula (V-a):

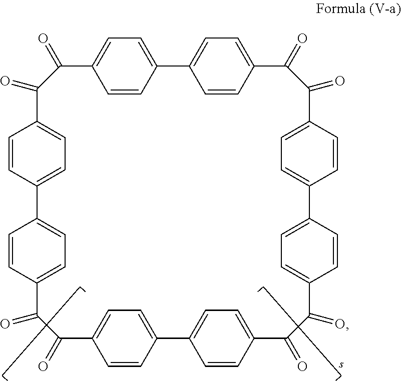

wherein:
s is 0, 1, 2, 3, or 4.

Embodiment 3A. The electrode of embodiment 1A, wherein the compound of Formula (I) is:

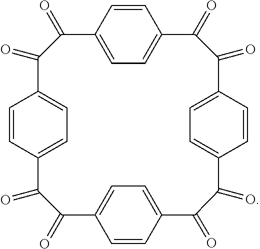 [SMILES: O=C1C(=O)c2ccc(cc2)C(=O)C(=O)c2ccc(cc2)C(=O)C(=O)c2ccc(cc2)C(=O)C(=O)c2ccc1cc2]

Embodiment 4A. The electrode of embodiment 1A, wherein the compound of Formula (III) is:

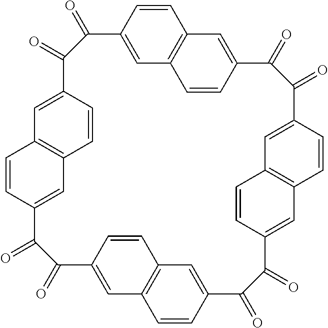 [SMILES: O=C1C(=O)c2ccc3cc(ccc3c2)C(=O)C(=O)c2ccc3cc(ccc3c2)C(=O)C(=O)c2ccc3cc(ccc3c2)C(=O)C(=O)c2ccc3cc1ccc3c2]

Embodiment 5A. The electrode of embodiment 1A, wherein the compound of Formula (V) is:

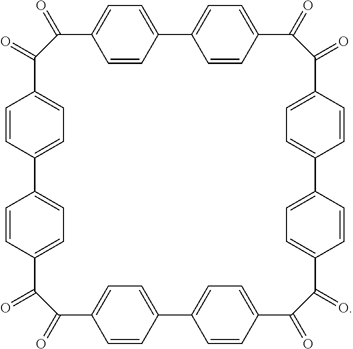 [SMILES: O=C1C(=O)c2ccc(cc2)-c2ccc(cc2)C(=O)C(=O)c2ccc(cc2)-c2ccc(cc2)C(=O)C(=O)c2ccc(cc2)-c2ccc(cc2)C(=O)C(=O)c2ccc(cc2)-c2ccc1cc2]

Embodiment 6A. The electrode of embodiment 1A, wherein the electrode is a cathode electrode.

Embodiment 7A. The electrode of embodiment 6A, wherein the cathode electrode is for a battery.

Embodiment 8A. The electrode of embodiment 7A, wherein the battery is a lithium-ion battery.

Embodiment 9A. The electrode of embodiment 1A, wherein the electrode further comprises at least one of: lithium, a compound comprising lithium, a lithium salt, or a lithium-ion.

Embodiment 10A. A lithium-ion battery, comprising: an anode electrode, a separator, an electrolyte, and the cathode electrode of embodiment 6A.

Embodiment 11A. The lithium ion-battery of embodiment 10A, wherein the electrolyte comprises at least one of: lithium, a compound comprising lithium, a lithium salt, or a lithium-ion.

Embodiment 12A. The electrode of embodiment 3A, wherein the electrode is a cathode electrode.

Embodiment 13A. A lithium-ion battery, comprising: an anode electrode, a separator, an electrolyte, and the cathode electrode of embodiment 12A.

Embodiment 14A. The lithium ion-battery of embodiment 13A, wherein the electrolyte comprises at least one of: lithium, a compound comprising lithium, a lithium salt, or a lithium-ion.

Embodiment 15A. The electrode of embodiment 4A, wherein the electrode is a cathode electrode.

Embodiment 16A. A lithium-ion battery, comprising: an anode electrode, a separator, an electrolyte, and the cathode electrode of embodiment 15A.

Embodiment 17A. The lithium ion-battery of embodiment 16A, wherein the electrolyte comprises at least one of: lithium, a compound comprising lithium, a lithium salt, or a lithium-ion.

Embodiment 18A. The electrode of embodiment 5A, wherein the electrode is a cathode electrode.

Embodiment 19A. A lithium-ion battery, comprising: an anode electrode, a separator, an electrolyte, and the cathode electrode of embodiment 18A.

Embodiment 20A. The lithium ion-battery of embodiment 19A, wherein the electrolyte comprises at least one of: lithium, a compound comprising lithium, a lithium salt, or a lithium-ion.

Additional Embodiments include those listed below:

Embodiment 1B. A composition, comprising at least one compound, wherein the at least one compound is selected from the group consisting of a compound of Formula (I), a compound of Formula (III), and a compound of Formula (V), and any combinations thereof; wherein the compound of Formula (I) is:

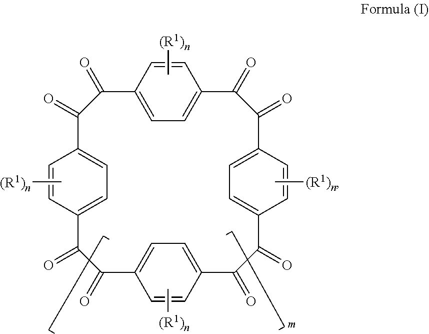

wherein:
m is 0, 1, 2, 3, or 4;
n is 0, 1, 2, 3 or 4; and
R¹ is an electron withdrawing group, or an electron donating group;
   wherein the compound of Formula (III) is:

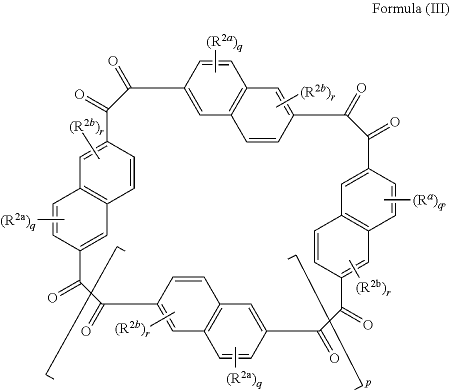

wherein:
p is 0, 1, 2, 3, or 4;
q is 0, 1, 2, or 3;
r is 0, 1, 2, or 3;
R^{2a} is an electron withdrawing group, or an electron donating group; and
R^{2b} is an electron withdrawing group, or an electron donating group; and
   wherein the compound of Formula (V) is:

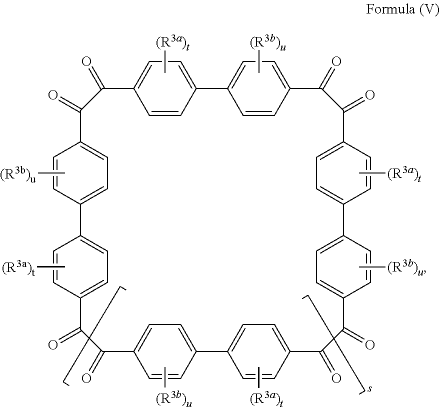

wherein:
s is 0, 1, 2, 3, or 4;
t is 0, 1, 2, 3 or 4;
u is 0, 1, 2, 3, or 4;
R^{3a} is an electron withdrawing group, or an electron donating group; and
R^{3b} is an electron withdrawing group, or an electron donating group.

Embodiment 2B. The composition of embodiment 1B, wherein the compound of Formula (I) is a compound of Formula (I-a):

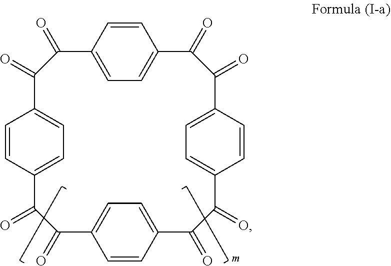

wherein:
m is 0, 1, 2, 3, or 4;
   wherein the compound of Formula (III) is a compound of Formula (III-a):

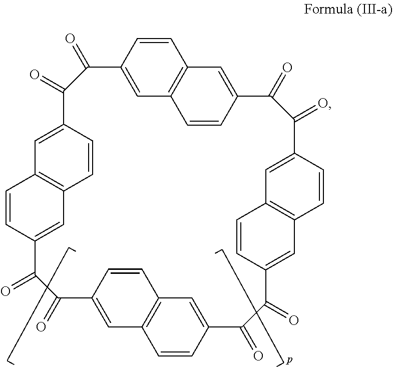

wherein:
p is 0, 1, 2, 3, or 4; and
   wherein the compound of Formula (V) is a compound of Formula (V-a):

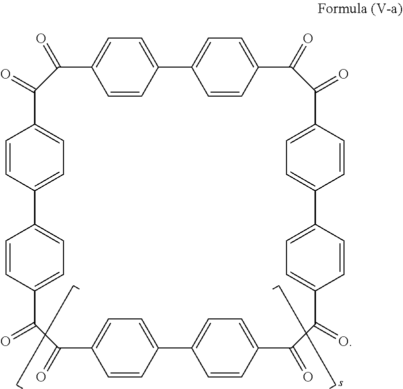

wherein:
s is 0, 1, 2, 3, or 4.

Embodiment 3B. The composition of embodiment 1B, wherein the compound of Formula (I) is:

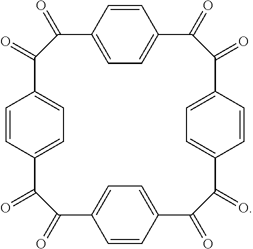 [SMILES: O=C1C(=O)c2ccc(cc2)C(=O)C(=O)c2ccc(cc2)C(=O)C(=O)c2ccc(cc2)C(=O)C(=O)c2ccc1cc2]

Embodiment 4B. The composition of embodiment 1B, wherein the compound of Formula (III) is:

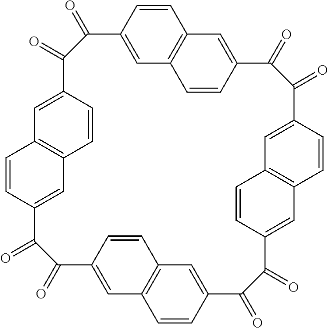 [SMILES: O=C1C(=O)c2ccc3cc(ccc3c2)C(=O)C(=O)c2ccc3cc(ccc3c2)C(=O)C(=O)c2ccc3cc(ccc3c2)C(=O)C(=O)c2ccc3cc1ccc3c2]

Embodiment 5B. The composition of embodiment 1B, wherein the compound of Formula (V) is:

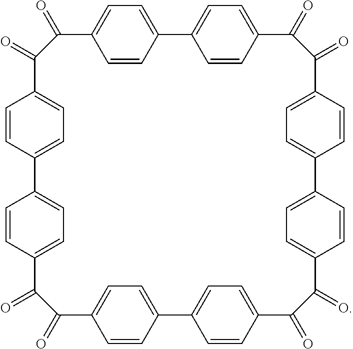 [SMILES: O=C1C(=O)c2ccc(cc2)-c2ccc(cc2)C(=O)C(=O)c2ccc(cc2)-c2ccc(cc2)C(=O)C(=O)c2ccc(cc2)-c2ccc(cc2)C(=O)C(=O)c2ccc(cc2)-c2ccc1cc2]

In some embodiments, the compound of Formula (I) is not

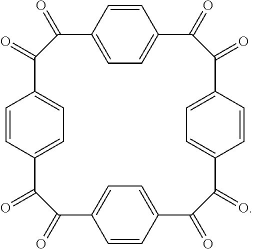 [SMILES: O=C1C(=O)c2ccc(cc2)C(=O)C(=O)c2ccc(cc2)C(=O)C(=O)c2ccc(cc2)C(=O)C(=O)c2ccc1cc2]

In some embodiments, the compound of Formula (I-a) is not

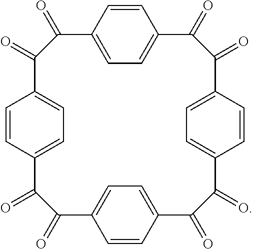 [SMILES: O=C1C(=O)c2ccc(cc2)C(=O)C(=O)c2ccc(cc2)C(=O)C(=O)c2ccc(cc2)C(=O)C(=O)c2ccc1cc2]

Additional Embodiments include those listed below:

Embodiment 1C. A compound of Formula (I):

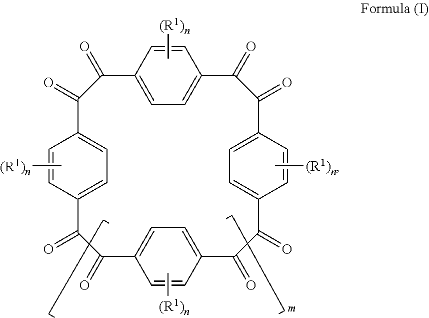

wherein:
m is 0, 1, 2, 3, or 4;
n is 0, 1, 2, 3 or 4; and
R¹ is an electron withdrawing group, or an electron donating group.

Embodiment 2C. The compound of embodiment 1C, wherein the compound of Formula (I) is a compound of Formula (I-a):

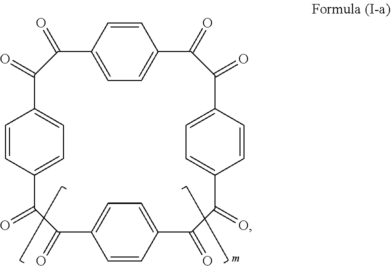

wherein:
m is 0, 1, 2, 3, or 4.

Embodiment 3C. The compound of embodiment 1C, wherein the compound of Formula (I) is:

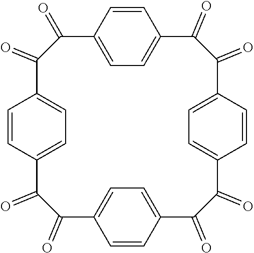 [SMILES: O=C1C(=O)c2ccc(cc2)C(=O)C(=O)c2ccc(cc2)C(=O)C(=O)c2ccc(cc2)C(=O)C(=O)c2ccc1cc2]

Embodiment 4C. The compound of embodiment 1C, wherein the compound of Formula (I) is not

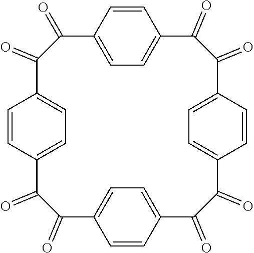 [SMILES: O=C1C(=O)c2ccc(cc2)C(=O)C(=O)c2ccc(cc2)C(=O)C(=O)c2ccc(cc2)C(=O)C(=O)c2ccc1cc2]

Embodiment 5C. The compound of embodiment 2C, wherein the compound of Formula (I-a) is not

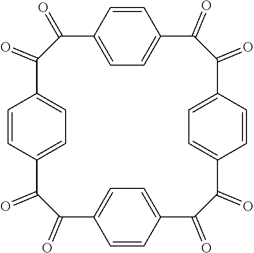 [SMILES: O=C1C(=O)c2ccc(cc2)C(=O)C(=O)c2ccc(cc2)C(=O)C(=O)c2ccc(cc2)C(=O)C(=O)c2ccc1cc2]

Additional Embodiments include those listed below:

Embodiment 1D. A compound of Formula (III):

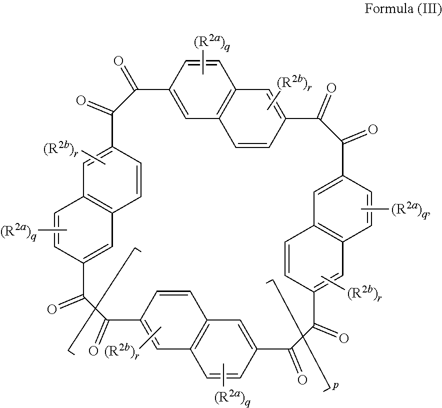

wherein:
p is 0, 1, 2, 3, or 4;
q is 0, 1, 2, or 3;
r is 0, 1, 2, or 3;
R^{2a} is an electron withdrawing group, or an electron donating group; and
R^{2b} is an electron withdrawing group, or an electron donating group; and

Embodiment 2D. The compound of embodiment 1D, wherein the compound of Formula (III) is a compound of Formula (III-a):

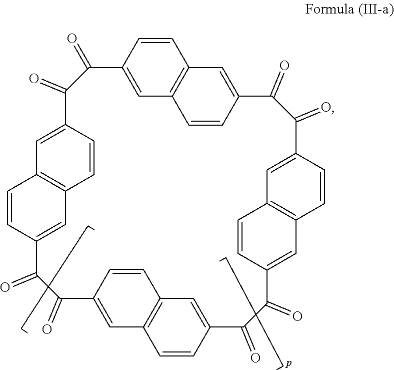

wherein:
p is 0, 1, 2, 3, or 4.

Embodiment 3D. The compound of embodiment ID, wherein the compound of Formula (III) is:

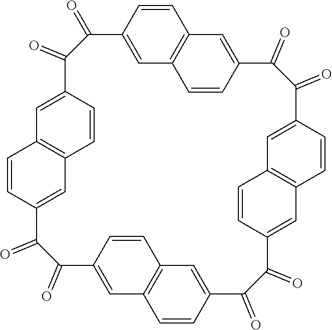 [SMILES: O=C1C(=O)c2ccc3cc(ccc3c2)C(=O)C(=O)c2ccc3cc(ccc3c2)C(=O)C(=O)c2ccc3cc(ccc3c2)C(=O)C(=O)c2ccc3cc1ccc3c2]

Additional Embodiments include those listed below:

Embodiment 1E. A compound of Formula (V):

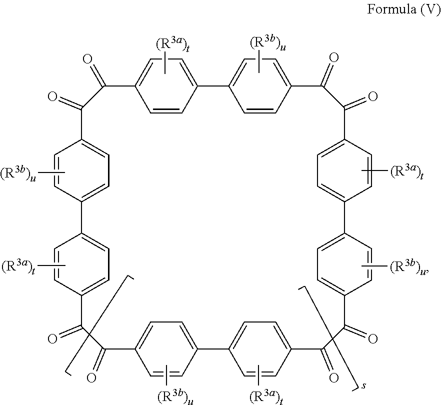

wherein:
s is 0, 1, 2, 3, or 4;
t is 0, 1, 2, 3 or 4;
u is 0, 1, 2, 3, or 4;
R^{3a} is an electron withdrawing group, or an electron donating group; and
R^{3b} is an electron withdrawing group, or an electron donating group.

Embodiment 2E. The compound of embodiment 1E, wherein the compound of Formula (V) is a compound of Formula (V-a):

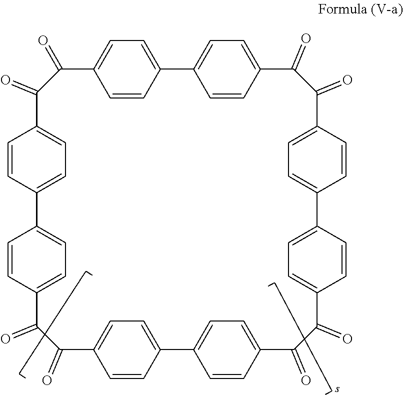

wherein:
s is 0, 1, 2, 3, or 4.

Embodiment 3E. The compound of embodiment 1E, wherein the compound of Formula (V) is:

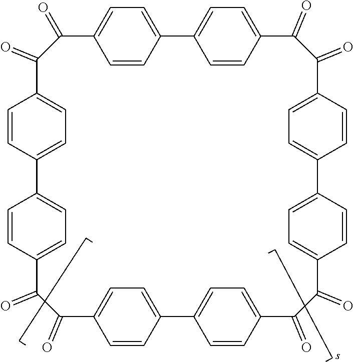

Additional Embodiments include those listed below:

Embodiment 1F. An article of manufacture, comprising at least one compound, wherein the at least one compound is selected from the group consisting of a compound of Formula (I), a compound of Formula (III), and a compound of Formula (V), and any combinations thereof;

wherein the compound of Formula (I) is:

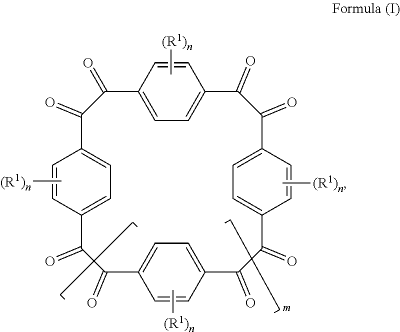

wherein:
m is 0, 1, 2, 3, or 4;
n is 0, 1, 2, 3 or 4; and
R¹ is an electron withdrawing group, or an electron donating group;
   wherein the compound of Formula (III) is:

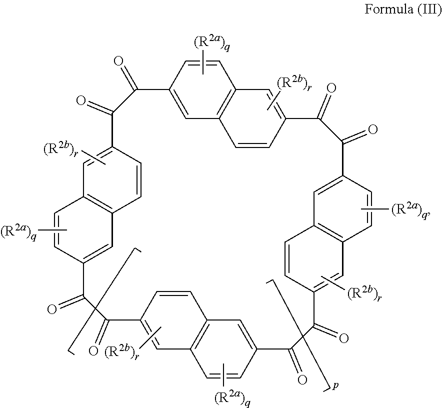

wherein:
p is 0, 1, 2, 3, or 4;
q is 0, 1, 2, or 3;
r is 0, 1, 2, or 3;
R^{2a} is an electron withdrawing group, or an electron donating group; and
R^{2b} is an electron withdrawing group, or an electron donating group; and
   wherein the compound of Formula (V) is:

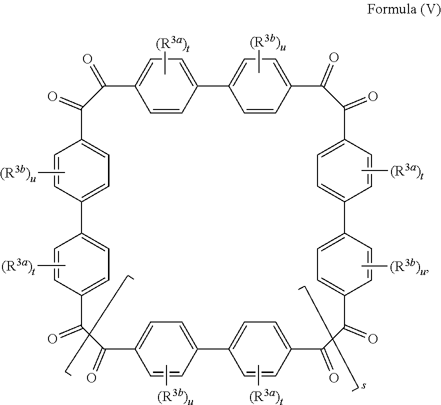

wherein:
s is 0, 1, 2, 3, or 4;
t is 0, 1, 2, 3 or 4;
u is 0, 1, 2, 3, or 4;
R^{3a} is an electron withdrawing group, or an electron donating group; and
R^{3b} is an electron withdrawing group, or an electron donating group.

Embodiment 2F. The article of manufacture of embodiment 1F, wherein the compound of Formula (I) is a compound of Formula (I-a):

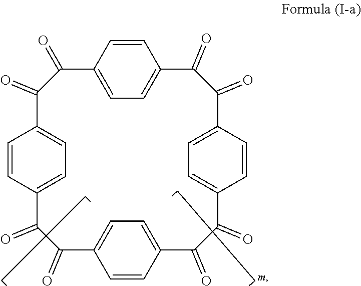

wherein:
m is 0, 1, 2, 3, or 4;
   wherein the compound of Formula (III) is a compound of Formula (III-a):

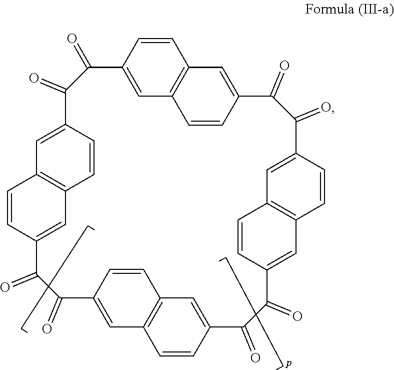

wherein:
p is 0, 1, 2, 3, or 4; and
   wherein the compound of Formula (V) is a compound of Formula (V-a):

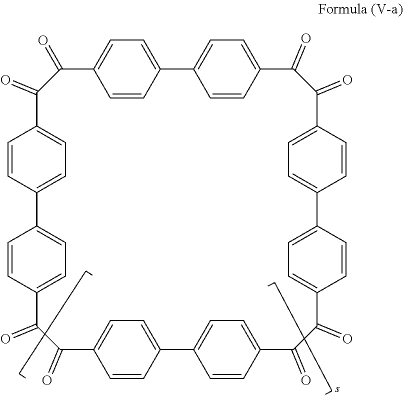

wherein:
s is 0, 1, 2, 3, or 4.

Embodiment 3F. The article of manufacture of embodiment 1F, wherein the compound of Formula (D) is:

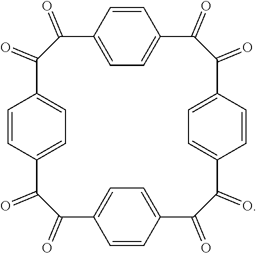 [SMILES: O=C1C(=O)c2ccc(cc2)C(=O)C(=O)c2ccc(cc2)C(=O)C(=O)c2ccc(cc2)C(=O)C(=O)c2ccc1cc2]

Embodiment 4F. The article of manufacture of embodiment 1F, wherein the compound of Formula (III) is.

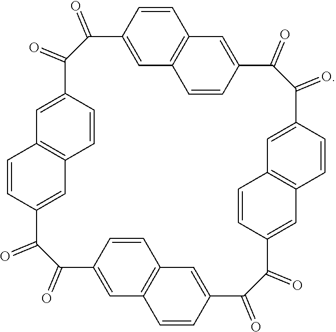 [SMILES: O=C1C(=O)c2ccc3cc(ccc3c2)C(=O)C(=O)c2ccc3cc(ccc3c2)C(=O)C(=O)c2ccc3cc(ccc3c2)C(=O)C(=O)c2ccc3cc1ccc3c2]

Embodiment 5F. The article of manufacture of embodiment 1F, wherein the compound of Formula (V) is:

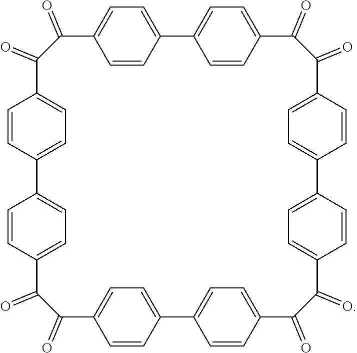 [SMILES: O=C1C(=O)c2ccc(cc2)-c2ccc(cc2)C(=O)C(=O)c2ccc(cc2)-c2ccc(cc2)C(=O)C(=O)c2ccc(cc2)-c2ccc(cc2)C(=O)C(=O)c2ccc(cc2)-c2ccc1cc2]

Embodiment 6F. The article of manufacture of embodiment 1F, wherein the article of manufacture is an electrode.

Embodiment 7F. The article of manufacture of embodiment 6F, wherein the electrode is a cathode electrode.

Embodiment 8F. The article of manufacture of embodiment 6F, wherein the electrode is for a battery.

Embodiment 9F. The article of manufacture of embodiment 7F, wherein the cathode electrode is for a battery.

Embodiment 10F. The article of manufacture of embodiment 8F, wherein the battery is a lithium-ion battery.

Embodiment 11F. The article of manufacture of embodiment 9F, wherein the battery is a lithium-ion battery.

Embodiment 12F. The article of manufacture of embodiment 6F, wherein the electrode comprises at least one of: lithium, a compound comprising lithium, a lithium salt, or a lithium-ion.

Embodiment 13F. The article of manufacture of embodiment 7F, wherein the cathode electrode comprises at least one of: lithium, a compound comprising lithium, a lithium salt, or a lithium-ion.

Additional Embodiments include those listed below:

In various embodiments, the present invention provides a battery, wherein the battery comprises a compound of Formula (I), a compound of Formula (I-a), a compound of Formula (I-b), a compound of Formula (III), a compound of Formula (III-a), a compound of Formula (III-b), a compound of Formula (V), a compound of Formula (V-a), a compound of Formula (V-b), or any combination thereof.

In various embodiments, the present invention provides an electrode, wherein the electrode comprises a compound of Formula (I), a compound of Formula (I-a), a compound of Formula (I-b), a compound of Formula (III), a compound of Formula (III-a), a compound of Formula (III-b), a compound of Formula (V), a compound of Formula (V-a), a compound of Formula (V-b), or any combination thereof.

In various embodiments, the present invention provides a lithium-storage device, wherein the lithium storage device comprises a compound of Formula (I), a compound of Formula (I-a), a compound of Formula (I-b), a compound of Formula (III), a compound of Formula (III-a), a compound of Formula (III-b), a compound of Formula (V), a compound of Formula (V-a), a compound of Formula (V-b), or any combination thereof.

In various embodiments, the present invention provides a composition, wherein the composition comprises a compound of Formula (I), a compound of Formula (I-a), a compound of Formula (I-b), a compound of Formula (III), a compound of Formula (III-a), a compound of Formula (III-b), a compound of Formula (V), a compound of Formula (V-a), a compound of Formula (V-b), or any combination thereof.

In some embodiments, the present invention provides an article of manufacture, wherein the article of manufacture comprises a compound of Formula (I), a compound of Formula (I-a), a compound of Formula (I-b), a compound of Formula (III), a compound of Formula (III-a), a compound of Formula (III-b), a compound of Formula (V), a compound of Formula (V-a), a compound of Formula (V-b), or any combination thereof.

In various embodiments, the present invention provides a compound of Formula (I), a compound of Formula (I-a), a compound of Formula (I-b), a compound of Formula (III), a compound of Formula (III-a), a compound of Formula (III-b), a compound of Formula (V), a compound of Formula (V-a), or a compound of Formula (V-b).

In various embodiments, the present invention provides a compound of Formula (I), a compound of Formula (I-a), a compound of Formula (I-b), a compound of Formula (III), a compound of Formula (III-a), a compound of Formula (III-b), a compound of Formula (V), a compound of Formula (V-a), or a compound of Formula (V-b), with the proviso that the compound of Formula (I) is not

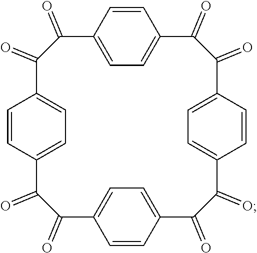 [SMILES: O=C1C(=O)c2ccc(cc2)C(=O)C(=O)c2ccc(cc2)C(=O)C(=O)c2ccc(cc2)C(=O)C(=O)c2ccc1cc2]

with the proviso that the compound of Formula (I-a) is not

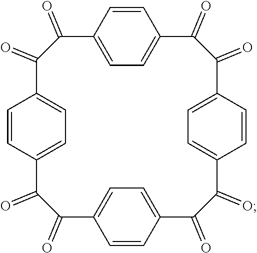 [SMILES: O=C1C(=O)c2ccc(cc2)C(=O)C(=O)c2ccc(cc2)C(=O)C(=O)c2ccc(cc2)C(=O)C(=O)c2ccc1cc2]

and with the proviso that the compound of Formula (I-b) is not

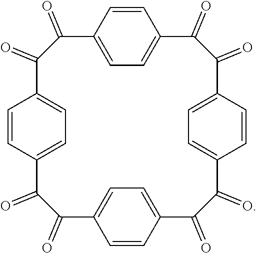 [SMILES: O=C1C(=O)c2ccc(cc2)C(=O)C(=O)c2ccc(cc2)C(=O)C(=O)c2ccc(cc2)C(=O)C(=O)c2ccc1cc2]

In various embodiments, the present invention provides a complex of the present invention.

In various embodiments, the present invention provides a battery, wherein the battery comprises a complex of the present invention.

In various embodiments, the present invention provides an electrode, wherein the electrode comprises a complex of the present invention.

In various embodiments, the present invention provides a lithium-storage device, wherein the lithium-storage device comprises a complex of the present invention.

In various embodiments, the present invention provides a composition, wherein the composition comprises a complex of the present invention.

In various embodiments, the present invention provides an article of manufacture, wherein the article of manufacture comprises a complex of the present invention.

In some embodiments, the composition of the present invention is a composition for lithium-ion storage. In some embodiments, the article of manufacture of the present invention is an article of manufacture for lithium-ion storage.

In some embodiments, the electrode is a cathode. In some embodiments, the cathode is a cathode electrode. In some embodiments, the electrode is a cathode electrode. In some embodiments, the electrode is an organic electrode.

In some embodiments, the electrode is an anode. In some embodiments, the anode is an anode electrode. In some embodiments, the electrode is an anode electrode.

In some embodiments the battery is a lithium-ion battery.

In some embodiments, the electrode comprises at least one of: lithium, a compound comprising lithium, a lithium salt, or a lithium-ion.

In various embodiments, the present invention provides a battery comprising: an anode electrode, a separator, an electrolyte, and the cathode electrode.

In various embodiments, the present invention provides a lithium-ion battery comprising: an anode electrode, a separator, an electrolyte, and the cathode electrode.

In some embodiments, the separator comprises glass fibers. In some embodiments, the separator is a glass fiber separator. In some embodiments, the separator comprises a polyolefin. In some embodiments, non-limiting examples of a polyolefin include polyethylene, or polypropylene. In some embodiments, additional non-limiting examples of separators for use in the present invention are those known in the art.

In various embodiments, the compounds of the present invention (e.g., compounds of Formula (I), compounds of Formula (I-a), a compound of Formula (I-b), compounds of Formula (III), compounds of Formula (III-a), a compound of Formula (III-b), compounds of Formula (V), compounds of Formula (V-a), and/or a compound of Formula (V-b) have high per-gram capacity, are inexpensive, and have strong cycling performance.

In some embodiments, the electrolyte comprises at least one of: lithium, a compound comprising lithium, a lithium salt, or a lithium-ion. In some embodiments, non-limiting examples of an electrolyte or lithium salt include lithium hexafluorophosphate (LiPF₆), lithium bis(trifluoromethanesulfonyl)imide (LiTFSI), lithium perchlorate (LiClO₄), lithium tetrafluoroborate (LiBF₄), and lithium bis(trifluoromethanesulfonyl)imide (LiC₂F₆NO₄S₂). In some embodiments, additional non-limiting examples of an electrolyte or lithium salt for use in the present invention are those known in the art.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Experimental—General Methods and Materials

All syntheses were performed under nitrogen in standard oven-dried glassware. ¹H and ¹³C NMR spectra were recorded on JEOL ECA-500 MHz spectrometers with working frequencies of 500 MHz for ¹H and 126 MHz for ¹³C. ¹³C NMR spectra were recorded with simultaneous decoupling of H nuclei. Chemical shifts are reported in ppm (δ) with the residual signals of deuterated solvents used for calibration. Infrared spectra were collected on a Nicolet iS10 FT-IR spectrometer. Mass spectrometry analyses were performed by the Mass Spectrometry Facility at University of Texas-Austin. Melting points were measured in open capillary tubes using a Barnstead International Mel-TEMP apparatus and are uncorrected.

The following starting materials and solvents were purchased from commercial suppliers and used without further purification: Dess-Martin periodinane, NaCN, and CH₂Cl₂ from Sigma Aldrich; 2-naphthaldehyde, copper(II) sulfate pentahydrate, NEt₃, acetone, and THF from Oakwood Chemicals; 3-ethyl-5-(2-hydroxyethyl)-4-methylthiazolium bromide and pyridine from Alfa Aesar; absolute EtOH from Deacon Laboratories. The 4 Å molecular sieves used to dry THF were purchased from ThermoFisher and activated in a vacuum oven at 120° C. overnight.

Syntheses of Compound 1, Compounds 5a/5b, and Compounds 6a/6b

Synthesis of cyclotetrabenzil compound 1, cyclotetrabenzoin compounds 5a/5b and cyclotetrabenzoin compounds 6a/6b were synthesized following literature methods with slight modifications.

### Synthesis of Cyclotetrabenzil Macrocycle Compound 2

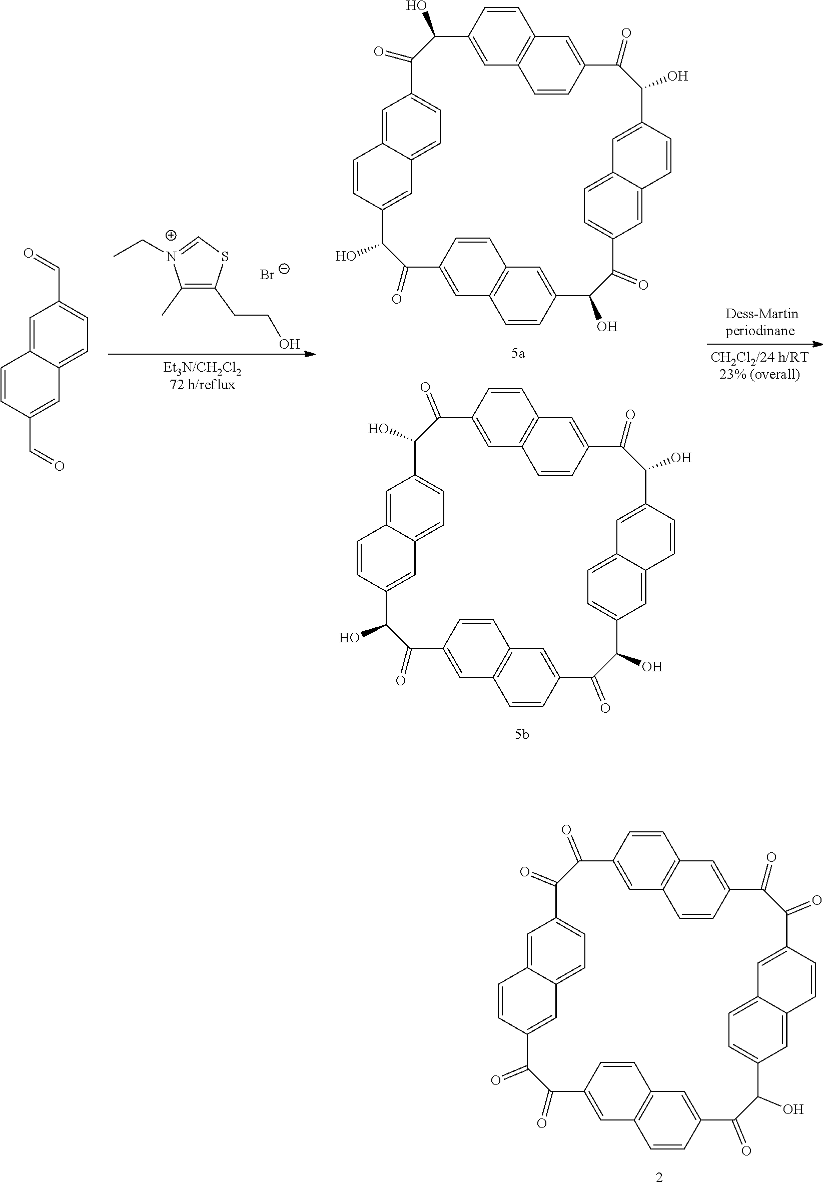 [SMILES: O=Cc1ccc2cc(C=O)ccc2c1 ; CC[n+]1csc(CCO)c1C ; [Br-] ; O=C1c2ccc3cc(ccc3c2)[C@@H](O)C(=O)c2ccc3cc(ccc3c2)[C@H](O)C(=O)c2ccc3cc(ccc3c2)[C@@H](O)C(=O)c2ccc3cc(ccc3c2)[C@@H]1O ; O=C1c2ccc3cc(ccc3c2)C(=O)[C@H](O)c2ccc3cc(ccc3c2)[C@@H](O)C(=O)c2ccc3cc(ccc3c2)C(=O)[C@@H](O)c2ccc3cc(ccc3c2)[C@@H]1O ; O=C1C(=O)c2ccc3cc(ccc3c2)C(=O)C(=O)c2ccc3cc(ccc3c2)C(O)C(=O)c2ccc3cc(ccc3c2)C(=O)C(=O)c2ccc3cc1ccc3c2]

The crude mixture of cyclotetrabenzoin compound 5a and cyclotetrabenzoin compound 5b was suspended in dry CH₂Cl₂ (30 mL) in a 100 mL two-neck round bottom flask equipped with a magnetic stirrer. The suspension was cooled to 0° C. under an N₂ atmosphere and Dess-Martin periodinane (6.70 g, 5.17 mmol) was slowly added to the reaction mixture. The reaction was warmed to room temperature and stirred under N₂ for 24 h. The yellow precipitate was collected through filtration and washed with saturated aq. solution of NaHCO₃ (3×20 mL). The dried solid was collected and recrystallized from acetone to afford pure compound 2 as a crystalline yellow powder in 23% overall yield (0.64 g, 0.87 mmol).

Compound 2: mp >360° C., decomposition. ¹H NMR (500 MHz, DMSO-d₆) δ 8.53 (s, 8H), 8.18 (d, J=8.5 Hz, 8H), 7.77 (d, J==8 Hz, 8H) ppm. ¹³C NMR (126 MHz, DMSO-d₆) δ 196.16, 135.52, 132.47, 132.23, 125.52 ppm. FT-IR (neat): \tilde{v}=1674, 1590, 1407, 1355, 1238, 1160, 1118, 849, 679, 633, 620, 605 cm⁻¹. ESI-HRMS: m/z [M+Na]⁺: Calcd for [C₄₈H₂₄O₈·Na]⁺751.1363; Found 751.1358, with correct isotope distribution.

### Synthesis of Cyclotetrabenzil Macrocycle Compound 3

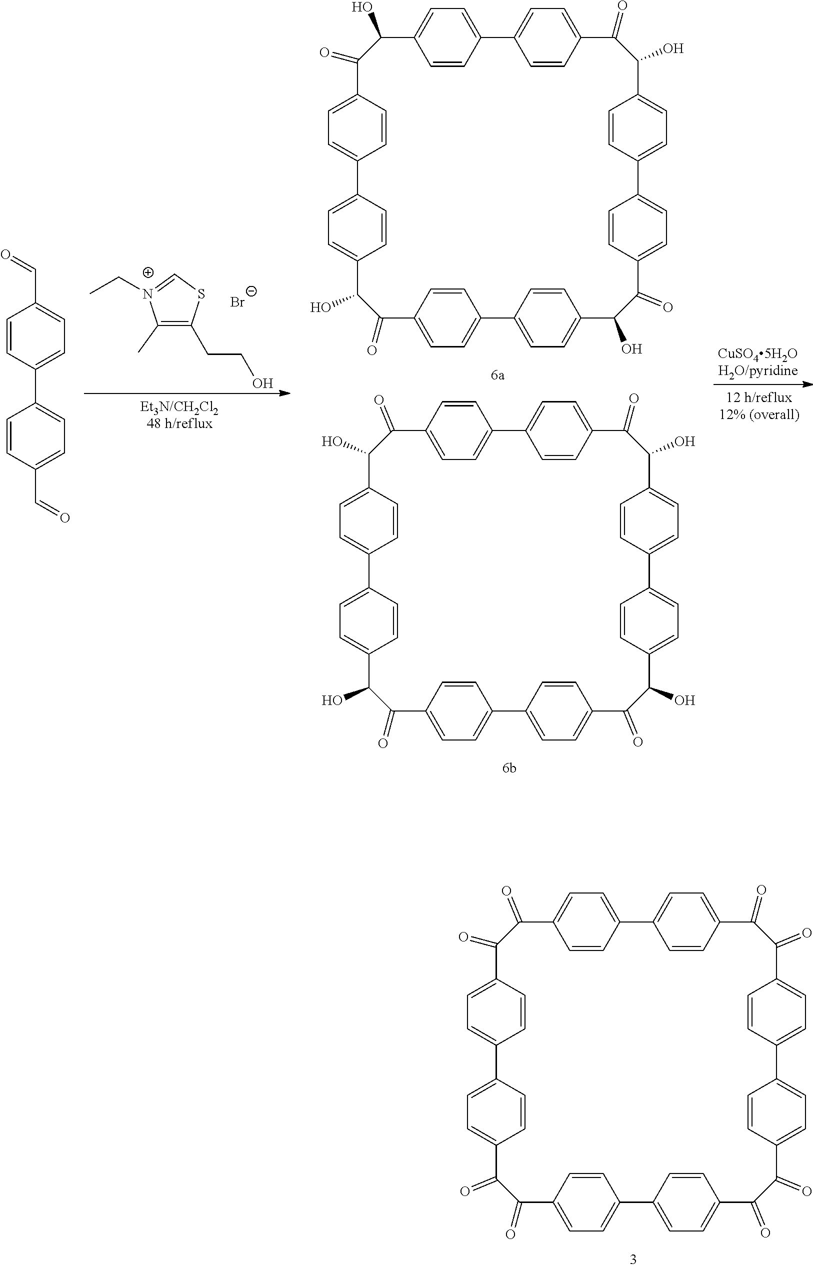 [SMILES: O=Cc1ccc(-c2ccc(C=O)cc2)cc1 ; CC[n+]1csc(CCO)c1C ; [Br-] ; O=C1c2ccc(cc2)-c2ccc(cc2)[C@@H](O)C(=O)c2ccc(cc2)-c2ccc(cc2)[C@H](O)C(=O)c2ccc(cc2)-c2ccc(cc2)[C@@H](O)C(=O)c2ccc(cc2)-c2ccc(cc2)[C@@H]1O ; O=C1c2ccc(cc2)-c2ccc(cc2)C(=O)[C@H](O)c2ccc(cc2)-c2ccc(cc2)[C@@H](O)C(=O)c2ccc(cc2)-c2ccc(cc2)C(=O)[C@@H](O)c2ccc(cc2)-c2ccc(cc2)[C@@H]1O ; O=C1C(=O)c2ccc(cc2)-c2ccc(cc2)C(=O)C(=O)c2ccc(cc2)-c2ccc(cc2)C(=O)C(=O)c2ccc(cc2)-c2ccc(cc2)C(=O)C(=O)c2ccc(cc2)-c2ccc1cc2]

A solution of CuSO₄·5H₂O (0.81 g, 5.05 mmol) in H₂O (6 mL) and pyridine (2 mL) was prepared in a 25 mL two-neck round-bottom flask equipped with a magnetic stirrer and fitted with a condenser. The solution was heated to 60° C. until all solids dissolved, and then a mixture of crude cyclotetrabenzoin compound 6a and cyclotetrabenzoin compound 6b (0.50 g, 0.60 mmol) was slowly added to the reaction solution. The temperature was increased to reflux overnight. The heating was discontinued and the reaction mixture was allowed to cool to room temperature over 24 h without disturbance. The blue aqueous layer was decanted, and the remaining brown-yellow solid was washed with hot 10% aq. HCl (3×10 mL). The solid was then boiled in CHCl₃ (150 mL), cooled, and the entire mixture was added to a separatory funnel. The organic layer was washed with H₂O (3×10 mL), collected, and dried over anhydrous MgSO₄. The solution was then passed through a 5 cm-long Celite pad. The solvent was removed under pressure and the light brown solid recrystallized from acetone to afford pure 3 as a yellow powder in 12% overall yield (60 mg, 0.07 mmol).

Compound 3: mp 332-334° C. ¹H NMR (500 MHz, acetone-d₆) δ 8.14 (d. J=7.5 Hz, 16H), 7.87 (d, J=8.0 Hz, 16H) ppm. ¹³C NMR (126 MHz, DMSO-d₆) δ 193.70, 136.64, 129.93, 129.29, 124.70 ppm. FT-IR (neat): \tilde{v}=1672, 1560, 1412, 1287, 1112, 927, 831, 751, 655, 542, 472 cm⁻¹. NCI-HRMS: m/z [M]⁻: Calcd for [C₅₆H₃₂O₈] 832.2097; Found 832.2105, with correct isotope distribution.

Synthesis of 1,2-Bis(2-napthyl)ethan-1,2-dione (compound 4)

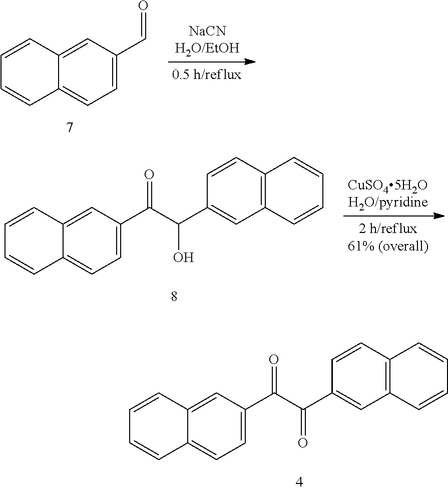 [SMILES: O=Cc1ccc2ccccc2c1 ; O=C(c1ccc2ccccc2c1)C(O)c1ccc2ccccc2c1 ; O=C(C(=O)c1ccc2ccccc2c1)c1ccc2ccccc2c1]

A solution of aldehyde 7 (0.50 g, 3.20 mmol) and NaCN (0.11 g, 2.14 mmol) in a mixture of H₂O (2 mL) and EtOH (4 mL) was prepared in a 50 mL round-bottom flask equipped with a magnetic stirrer and fitted with a condenser. The solution was heated at reflux for 30 min, then cooled to room temperature, and the precipitate was collected through vacuum filtration. The residue was washed with EtOH (20 mL) and dried under vacuum to afford crude benzoin 8 (402 mg) as a white powder which was used in the next reaction without further purification.

A solution of CuSO₄·5H₂O (0.12 g, 2.15 mmol) in a mixture of H₂O (6 mL) and pyridine (2 mL) was prepared in a 25 mL two-neck round-bottom flask equipped with a magnetic stirrer and fitted with a condenser. The solution was heated to 100° C. under an N₂ atmosphere until all solids dissolved. Then, crude product 8 was added into the reaction mixture and the solution was heated at reflux for 2 h. The heating was discontinued and the reaction mixture allowed to cool to room temperature overnight without disturbance. The blue aqueous layer was decanted, and the remaining yellow solid was washed with hot 10% aq. solution of HCl (3×10 mL). The solid was then dissolved in CH₂Cl₂ and added to a separatory funnel. The organic layer was washed with H₂O) (3×10 mL), collected, and dried over anhydrous MgSO₄. The solution was passed through a 5 cm Celite pad. The solvent was removed under reduced pressure and the orange solid was recrystallized from acetone to afford pure diketone compound 4 as a crystalline light-yellow powder in 63% overall yield (0.31 mg, 1.00 mmol).

Compound 4: ¹H NMR (500 MHz, CDCl₃) δ 8.45 (s, 2H), 8.15 (dd, J=8.5, 1.5 Hz, 2H), 7.99 (d, J=8.5 Hz, 2H), 7.91 (dd, J=8.0, 3.0 Hz, 4H), 7.63-7.66 (m, 2H), 7.55 (dd, J=8.0, 7.0 Hz, 2H) ppm.

### Cell Preparation and Electrochemical Measurements

All experiments were performed at room temperature, and all cells were assembled in an mBraun argon-filled glovebox (<0.5 ppm water and oxygen content). Electrochemical measurements were recorded on Bio-Logic VMP3 potentiostat and LAND CT-2001A battery tested. The following starting materials and solvents were purchased from commercial suppliers and used without further purification: lithium bis(trifluoromethanesulfonyl)imide (LiTFSI), dimethoxyethane (DME), and dioxolane (DOL) from MilliporeSigma. LiTFSI was allowed to dry in the glovebox for 24 prior to being used.

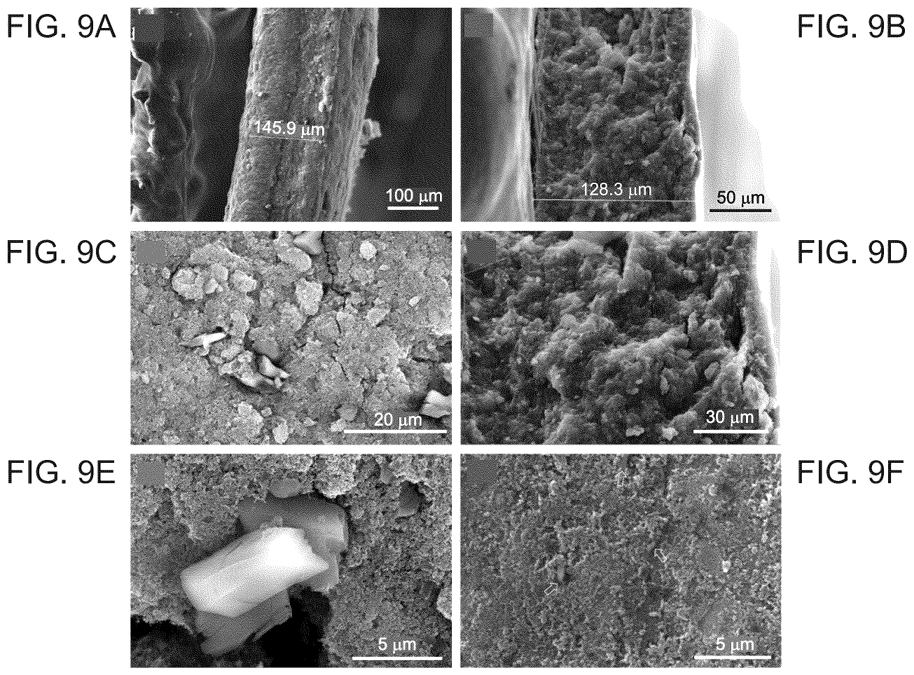

To fabricate free-standing electrodes, the active material (compounds 1-4), Katjenblack carbon (KB), and polytetrafluoroethylene (PTFE) binder were mixed in a 5:4:1 mass ratio and then pressed into pellets with the aid of EtOH (areal mass loading ˜1.5 mg cm⁻²). The typical thickness of these free-standing cathodes was ˜150 μm from the SEM measurement (FIG. 9A). For Fourier transform infrared measurements, macrocycle compound 2, KB, and PTFE binder were mixed in a 6:3:1 mass ratio to give an active material areal mass loading of ˜2 mg cm⁻² For the long cycling and rate performance test, a mass ratio of 3:5:2 (30% of compound 2 and compound 4) was used, and the mixture was rolled onto a stainless-steel mesh disc with ⅜-inch diameter (areal mass loading ˜0.4 mg cm⁻²). All electrodes were dried at 85° C. under vacuum for 24 hours before using.

The 0.5M lithium electrolyte solution was prepared by slowly adding LiTFSI (0.287 g) into a 1:1 solution of DME/DOL (2 mL). This 0.5M electrolyte solution was used for all electrochemical studies, while a more viscous 3M electrolyte solution (prepared following the same procedure as stated above) was used for performance characterization studies. CR2032 coin cell and glass fiber separators (Whatman, Grade GF/A) were used for all electrochemical characterizations unless otherwise indicated. Lithium foil with 7/16-inch diameter was used as the anode and the amount of electrolyte added was 60 μL. Half-cells were assembled adopting a conventional CR2032 coin cell architecture and subsequently using electrode 1, electrode 2, electrode 3, and electrode 4 as the cathode and lithium foil as the anode.

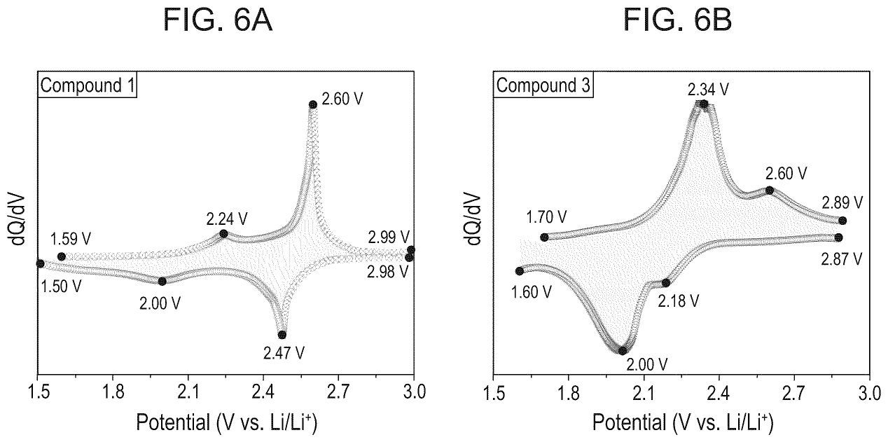

Macrocycle compound 1 and macrocycle compound 3 contain eight redox-active carbonyl groups-just like compound 2, and their dQ/dV profiles suggests, without being bound by theory, that they undergo a similar 2-step reversible redox process (FIG. 6A-FIG. 6B). However, as shown in FIG. 2A and FIG. 2C, a single molecule of compound 1 and compound 3 cannot reversibly store all eight electrons during a cycling process, suggesting, without being bound by theory, that not all redox sides are electrochemically active.

The charging/discharging profiles of compound 2 and compound 4 demonstrate much more rapid capacity decay of compound 4-within the first 10 cycles (FIG. 7A-FIG. 7B). This is further evident in the plots shown in FIG. 4.

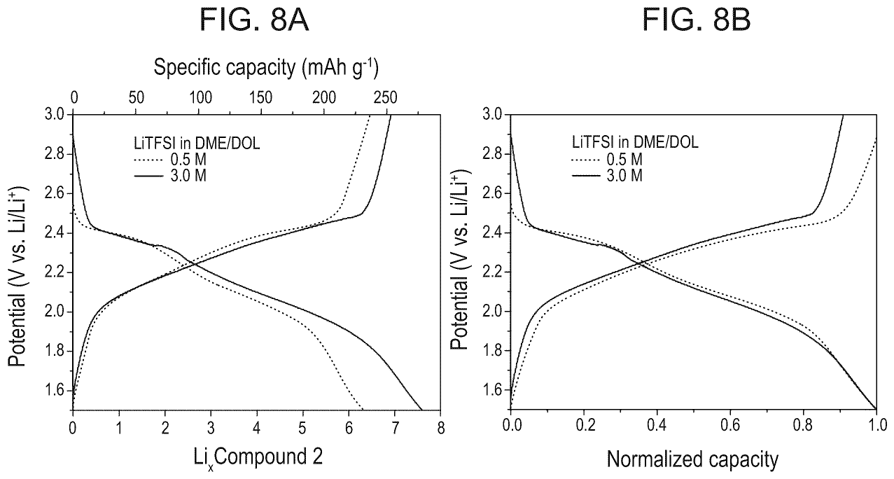

The cycling performance of compound 2 with different concentrations electrolytes is shown in FIG. 8A-FIG. 8B. The specific capacity approaches the 6.3 e⁻ transfer process at the low electrolyte concentration of 0.5M, but it increases up to 7.6 e⁻ as the electrolyte concentration increases to 3M. The normalized discharge-charge profiles obtained with different concentrations are almost identical, indicating no change in reaction mechanism. The decrease in capacity at low concentrations is thus ascribed to the result of kinetic limitations.

### Scanning Electron Microscopy (SEM) Images of the Electrode

Scanning electron microscopy was performed on powder samples using a JEOL JSM 6400 SEM instrument.

The SEM images of the pristine free-standing cathode 2 were shown in FIG. 9A, FIG. 9C, and FIG. 9E. The irregular polyhedron (compound 2) was wrapped by amorphous carbon (KB). After 12 cycles at a current density of 100 mA g⁻¹, the size of the polyhedron grains decreased and their distribution in the main carbon become more dispersed.

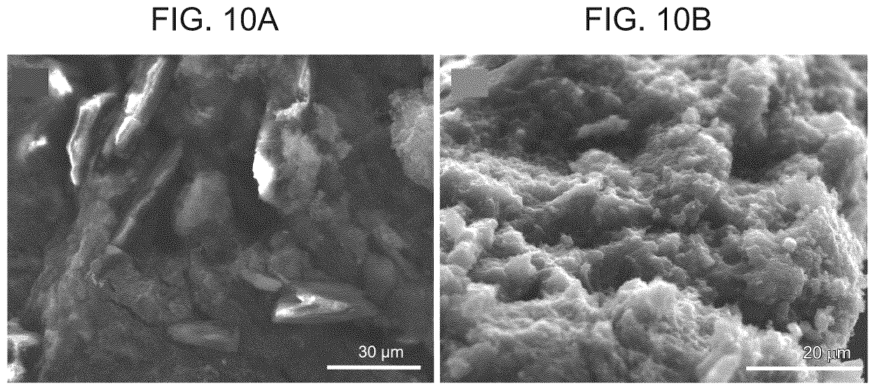

The SEM image of pristine cathode constructed from compound 4 is also shown in FIG. 10A, similar compound 4 polyhedral grains were surrounded by carbon. Under the same cycling condition, these grains almost disappeared while the dispersed carbon still remained in the structure.

### Solubility Tests of Compounds 1-4

In an argon-filled glovebox, certain mass of compounds 1-4 solids were added to vials. Then, 0.5M solution of LiTFSI in DME/DOL (v:v=1:1) was slowly added dropwise and the mixture was manually stirred until all material visibly dissolved. The amount of LiTFSI solution used was recorded to determine the solubility of each compound in the electrolyte. The order of solubility from highest to lowest is compound 4 (163.7 mmol L⁻¹), compound 1 (12.48 mmol L⁻¹), compound 2 (3.57 mmol L⁻¹), and compound 3 (2.76 mmol L⁻¹).

### UV-Vis Spectroscopy of Compounds 2 and 4

The CR2032 coin cell with cathode 2 or cathode 4 was discharged at the rate of 100 mA g⁻¹ until 50% state-of-discharge and was disassembled in an argon-filled glovebox. Then, the cathode and separator were immersed into 1.0 mL DME/DOL(1/1 v %) solvent for 6h. For the UV-vis absorption measurements, 30 μL of solution was extracted from the soaked vials and diluted with 3000 μL of DME/DOL solvent.

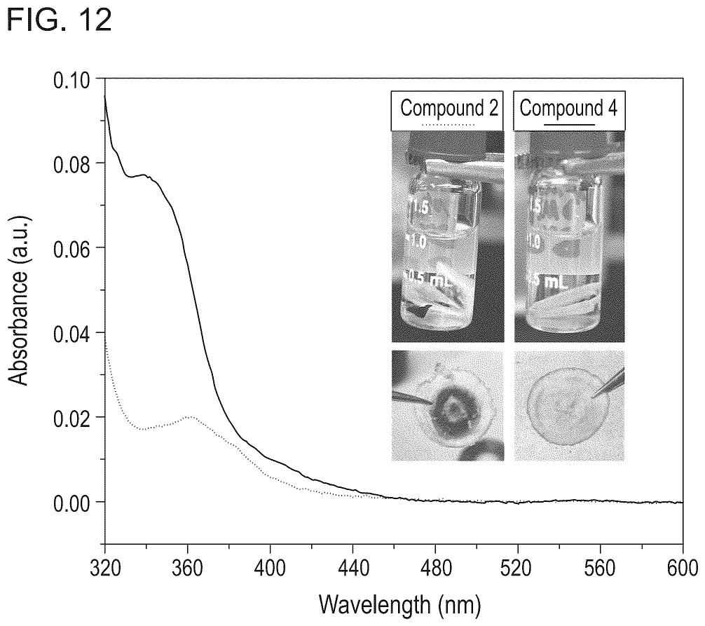

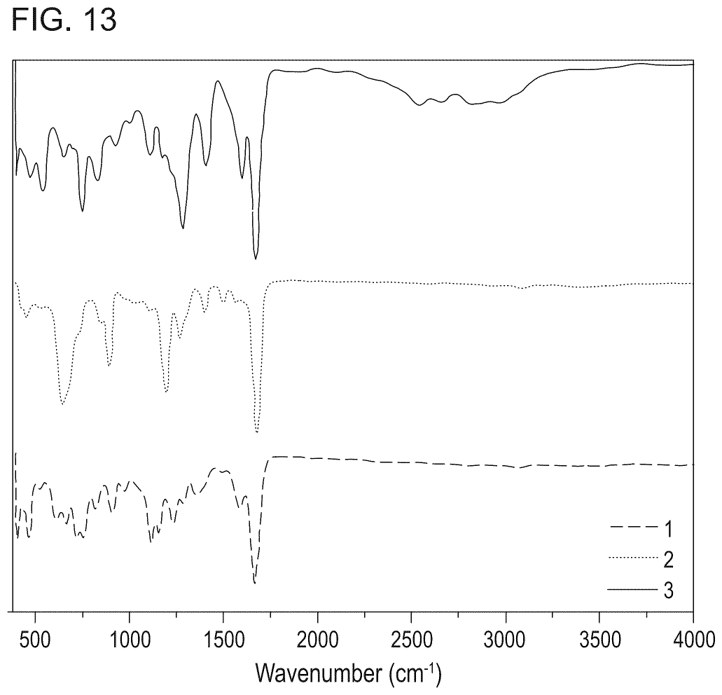

The optical images of the soaked solution and cathode/seperator combination are shown in the inset of FIG. 12. Solution of compound 4 appeared a distinct orange color. A comparison of UV-vis spectra demonstrated that the absorption peaks of soaked solution obtained from compound 4 and compound 2 are both at ˜360 nm. The absorbance intensity of compound 4 is 300% higher than that of compound 2, indicating a higher solubility of compound 4 at the 50% state-of-charge.

### IR Characterization

In situ infrared spectra were performed as single-reflection attenuated total reflectance measurements with powder samples using a Thermo Scientific Nicolet iS 5 instrument.

### DFT Calculations

Geometry optimizations were performed at the ωB97XD/def2-SVP level of theory employing the Gaussian 16 software package. Harmonic vibrational frequency analysis confirmed the nature of the stationary points. All optimized structures were confirmed as local minima with no imaginary frequencies. Attractive vs repulsive intermolecular interactions were evaluated using the non-covalent interaction (NCI) method.

Noncovalent interaction (NCI) plots for complex 2-Li, complex 2-Li₄, and complex 2-Li₈ reveal larger repulsive regions for complex 2-Li₈, as shown by the yellow-to-red colored surfaces. NCI plots are a density-based analysis that identify attractive and repulsive interactions (e.g., van der Waals interactions, steric clashes, hydrogen bonding) in a chemical system. Red surfaces indicate repulsive interactions, green surfaces indicate van der Waals interactions, and blue surfaces indicated attractive interactions. NCI analyses for compound 4 and complex 4-Li₂ show larger regions of yellow colored NCI surfaces in complex 4-Li₂ indicating increased repulsive interactions between the edge CC bonds of the naphthalene linkers. Reduced gradient isosurfaces were plotted using the Multiwfn program, red colored surfaces indicate repulsive interactions, green colored surfaces indicate van der Waals interactions, and blue colored surfaces indicate attractive interactions.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

What is claimed is:

1. An electrode, comprising at least one compound, wherein the at least one compound is selected from the group consisting of a compound of Formula (I), a compound of Formula (II), and a compound of Formula (V), and any combinations thereof;
wherein the compound of Formula (I) is: 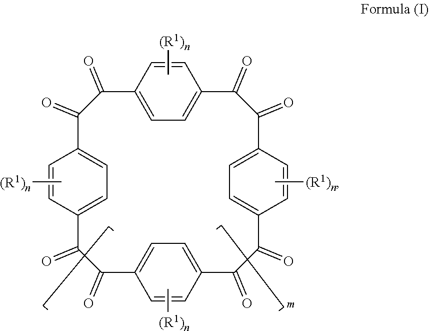
wherein:
m is 0, 1, 2, 3, or 4;
n is 0, 1, 2, 3 or 4; and
R¹ is an electron withdrawing group, or an electron donating group;
wherein the compound of Formula (II) is: 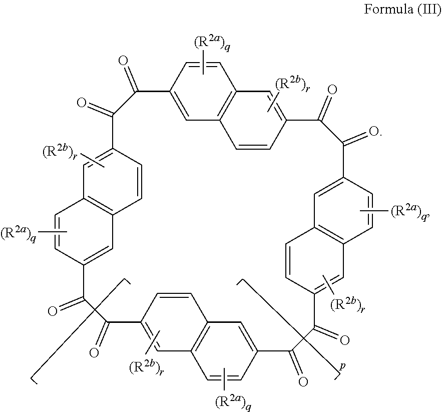
wherein:
p is 0, 1, 2, 3, or 4;
q is 0, 1, 2, or 3;
r is 0, 1, 2, or 3;
R^{2a} is an electron withdrawing group, or an electron donating group; and
R^{2b} is an electron withdrawing group, or an electron donating group; and
wherein the compound of Formula (V) is: 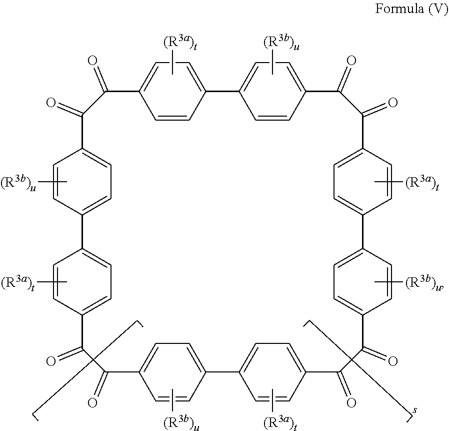
wherein:
s is 0, 1, 2, 3, or 4,
t is 0, 1, 2, 3 or 4;
u is 0, 1, 2, 3, or 4;
R^{3a} is an electron withdrawing group, or an electron donating group; and
R^{3b} is an electron withdrawing group, or an electron donating group.

2. The electrode of claim 1, wherein the compound of Formula (1) is a compound of Formula (I-a): 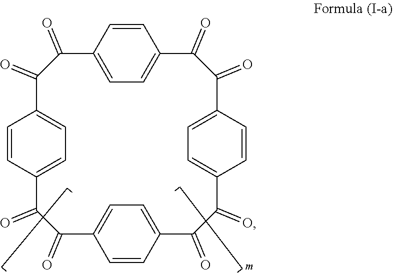
wherein:
m is 0, 1, 2, 3, or 4;
wherein the compound of Formula (III) is a compound of Formula (III-a): 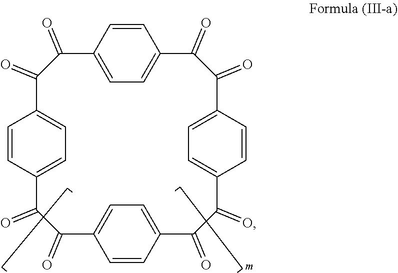
wherein:
p is 0, 1, 2, 3, or 4; and
wherein the compound of Formula (V) is a compound of Formula (V-a): 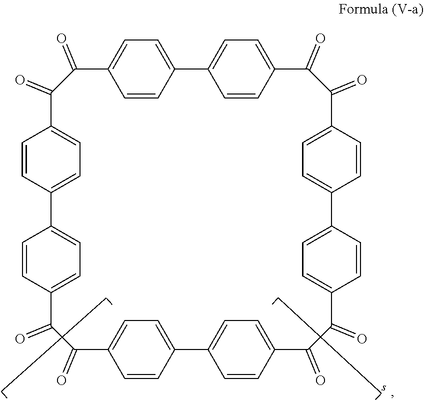
wherein:
s is 0, 1, 2, 3, or 4.

3. The electrode of claim 1, wherein the compound of Formula (I) is:

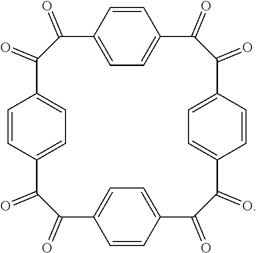 [SMILES: O=C1C(=O)c2ccc(cc2)C(=O)C(=O)c2ccc(cc2)C(=O)C(=O)c2ccc(cc2)C(=O)C(=O)c2ccc1cc2]

4. The electrode of claim 1, wherein the compound of Formula (III) is:

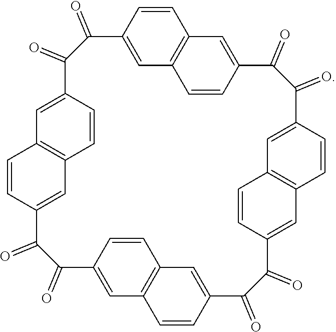 [SMILES: O=C1C(=O)c2ccc3cc(ccc3c2)C(=O)C(=O)c2ccc3cc(ccc3c2)C(=O)C(=O)c2ccc3cc(ccc3c2)C(=O)C(=O)c2ccc3cc1ccc3c2]

5. The electrode of claim 1, wherein the compound of Formula (V) is:

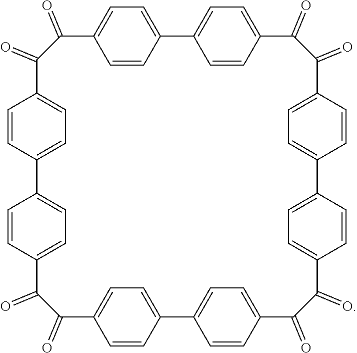 [SMILES: O=C1C(=O)c2ccc(cc2)-c2ccc(cc2)C(=O)C(=O)c2ccc(cc2)-c2ccc(cc2)C(=O)C(=O)c2ccc(cc2)-c2ccc(cc2)C(=O)C(=O)c2ccc(cc2)-c2ccc1cc2]

6. The electrode of claim 1, wherein the electrode is a cathode electrode.

7. The electrode of claim 6, wherein the cathode electrode is for a battery.

8. The electrode of claim 7, wherein the battery is a lithium-ion battery.

9. The electrode of claim 1, wherein the electrode further comprises at least one of: lithium, a compound comprising lithium, a lithium salt, or a lithium-ion.

10. A lithium-ion battery, comprising: an anode electrode, a separator, an electrolyte, and the cathode electrode of claim 6.

11. The lithium ion-battery of claim 10, wherein the electrolyte comprises at least one of: lithium, a compound comprising lithium, a lithium salt, or a lithium-ion.

12. The electrode of claim 3, wherein the electrode is a cathode electrode.

13. A lithium-ion battery, comprising: an anode electrode, a separator, an electrolyte, and the cathode electrode of claim 12.

14. The lithium ion-battery of claim 13, wherein the electrolyte comprises at least one of: lithium, a compound comprising lithium, a lithium salt, or a lithium-ion.

15. The electrode of claim 4, wherein the electrode is a cathode electrode.

16. A lithium-ion battery, comprising: an anode electrode, a separator, an electrolyte, and the cathode electrode of claim 15.

17. The lithium ion-battery of claim 16, wherein the electrolyte comprises at least one of: lithium, a compound comprising lithium, a lithium salt, or a lithium-ion.

18. The electrode of claim 5, wherein the electrode is a cathode electrode.

19. A lithium-ion battery, comprising: an anode electrode, a separator, an electrolyte, and the cathode electrode of claim 18.

20. The lithium ion-battery of claim 19, wherein the electrolyte comprises at least one of: lithium, a compound comprising lithium, a lithium salt, or a lithium-ion.
